SEQUENCE LISTING (32 sequences)

SEQ ID NO: 1 - protein, 288 aa, Homo sapiens
  REGION 1..288: note = human PD-1 including signal sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 113 aa, Homo sapiens [sequence omitted]

SEQ ID NO: 3 - protein, 107 aa, Homo sapiens [sequence omitted]

SEQ ID NO: 4 - protein, 120 aa, synthetic construct [sequence omitted]

SEQ ID NO: 5 - protein, 111 aa, synthetic construct [sequence omitted]

SEQ ID NO: 6 - protein, 121 aa, Mus musculus
QVQLQQSGAELAKPGASVQMSCKASGYSFTSSWIHWVKQRPGQGLEWIGYIYPSTGFTEYNQKFKDKATLTADKSSSTAYMQLSSLTSEDSAVYYCARWRDSSGYHAMDYWGQGTSVTVSS

SEQ ID NO: 7 - protein, 111 aa, Mus musculus
  REGION 1..111: note = Anti-PD-1 Antibody mAb 3 Light Chain Variable Region
DIVLTQSPASLTVSLGQRATISCRASQSVSTSGYSYMHWYQQKPGQPPKLLIKFGSNLESGIPARFSGSGSGTDFTLNIHPVEEEDTATYYCQHSWEIPYTFGGGTKLEIK

SEQ ID NO: 8 - protein, 117 aa, synthetic construct
  REGION 1..117: note = Humanized Anti-PD-1 Antibody mAb 4 Heavy Chain Variable Region
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 9 - protein, 106 aa, synthetic construct
  REGION 1..106: note = Humanized Anti-PD-1 Antibody mAb 4 Light Chain Variable Region
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 10 - protein, 525 aa, Homo sapiens
  REGION 1..525: note = human LAG3 (CD223) protein including signal sequence
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 11 - protein, 120 aa, Homo sapiens
  REGION 1..120: note = Anti-LAG-3 Antibody mAb 1 Heavy Chain Variable Region
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 12 - protein, 107 aa, Homo sapiens
  REGION 1..107: note = Anti-LAG-3 Antibody mAb 1 Light Chain Variable Region
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 13 - protein, 8 aa, synthetic construct
  REGION 1..8: note = Linker 1
GGGSGGGG

SEQ ID NO: 14 - protein, 28 aa, synthetic construct
  REGION 1..28: note = E-Coil Heterodimer-Promoting Domain
EVAALEKEVAALEKEVAALEKEVAALEK

SEQ ID NO: 15 - protein, 28 aa, synthetic construct
  REGION 1..28: note = K-Coil Heterodimer-Promoting Domain
KVAALKEKVAALKEKVAALKEKVAALKE

SEQ ID NO: 16 - protein, 28 aa, synthetic construct
  REGION 1..28: note = Cysteine-Containing E-Coil Heterodimer-Promoting Domain
EVAACEKEVAALEKEVAALEKEVAALEK

SEQ ID NO: 17 - protein, 28 aa, synthetic construct
  REGION 1..28: note = Cysteine-Containing K-Coil Heterodimer-Promoting Domain
KVAACKEKVAALKEKVAALKEKVAALKE

SEQ ID NO: 18 - protein, 6 aa, synthetic construct
  REGION 1..6: note = Cysteine-Containing Spacer Peptide
GGCGGG

SEQ ID NO: 19 - protein, 10 aa, synthetic construct
  REGION 1..10: note = Cysteine-Containing Linker Peptide
DKTHTCPPCP

SEQ ID NO: 20 - protein, 15 aa, synthetic construct
  REGION 1..15: note = Alternative Cysteine-Containing Linker Peptide
LEPKSADKTHTCPPC

SEQ ID NO: 21 - protein, 218 aa, Homo sapiens
  REGION 1..218: note = IgG1 Fc Region
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 218 aa, synthetic construct
  REGION 1..218: note = "Knob-Bearing" IgG1 CH2-CH3 Domain
PAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 218 aa, synthetic construct
  REGION 1..218: note = "Hole-Bearing" IgG1 CH2-CH3 Domain
PAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 24 - protein, 217 aa, synthetic construct
  REGION 1..217: note = IgG1 Fc Region L234A/L235A Variant
PAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 494 aa, synthetic construct
  REGION 1..494: note = First and Third Polypeptide Chains of the Exemplary PD-1 x LAG-3 Fc-DART-1 diabody
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKGGGSGGGGQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - DNA, 1482 bp, synthetic construct
  misc_feature 1..1482: note = Polynucleotide Encoding the First and Third Polypeptide Chains of the Exemplary PD-1 x LAG-3 Fc-DART-1 diabody
  source 1..1482: mol_type = other DNA
gaaattgtcctgacacagtctcccgcaaccctgagtttgagtcctggggagcgagcaactctctcctgccgagcctcccagagtatctcctcctacctcgcctggtaccaacagaagccagggcaggctccaaggctgcttatctatgacgcctctaaccgcgcaactgggattcccgcacgcttctccggctctggttccggcacagactttacacttactatctctagcctggagccagaagactttgccgtgtactattgtcagcaacgttccaattggccccttacctttgggcagggcactaacttggaaatcaaaggtggcggatccggcggcggaggccaggttcagctggtcgagagtggtggcggcgttgtgcaacctgggcgttccctccgattggactgtaaagcttccggcattactttctcaaattccggcatgcattgggtgaggcaagcccctggaaaagggctcgaatgggtggctgtgatttggtacgatggcagcaaacggtactacgccgattctgttaagggccgctttaccatctcccgcgataactcaaagaacacactgtttctgcaaatgaatagtcttagagccgaggacaccgccgtgtactactgtgccacaaatgacgattattgggggcagggcacattggtcacagtgtcttccggaggatgtggcggtggagaagtggccgcatgtgagaaagaggttgctgctttggagaaggaggtcgctgcacttgaaaaggaggtcgcagccctggagaaactggagcccaaatctgctgacaaaactcacacatgcccaccgtgcccagcacctgaagccgcggggggaccgtcagtcttcctcttccccccaaaacccaaggacaccctctatatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 27 - protein, 269 aa, synthetic construct
  REGION 1..269: note = Second and Fourth Polypeptide Chains of the Exemplary PD-1 x LAG-3 Fc-DART-1 diabody
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGSGGGGQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSGGCGGGKVAACKEKVAALKEKVAALKEKVAALKE

SEQ ID NO: 28 - DNA, 807 bp, synthetic construct
  misc_feature 1..807: note = Polynucleotide Encoding the Second and Fourth Polypeptide Chains of the Exemplary PD-1 x LAG-3 Fc-DART-1 diabody
  source 1..807: mol_type = other DNA
gagatcgtacttacccagtctcccgccaccctttccctgagtcctggtgagcgggccactctttcctgtcgcgcaagccaatcagtttctagctacctcgcatggtatcagcagaagccagggcaggcacccaggcttctcatctatgacgccagtaaccgcgcaaccgggatacctgctagattttccggcagtggatctgggaccgatttcacactgacaatttcatccttggaaccagaagatttcgcagtctactactgccagcaatcttccaactggccaagaactttcggacaggggaccaaagtggaaattaaaggtggcggatccggcggcggaggccaggtccagctccagcaatggggagccgggctgctgaaaccctctgaaacactgagtctcacatgtgccgtttatggaggttccttctccgattattactggaactggattcgtcagcctcccggcaagggcctggagtggatcggtgagattaaccacaatggcaataccaatagcaatcctagtttgaaatctcgcgtcactctttccctcgatacaagcaaaaaccagttttctttgaaattgcgatctgtaactgctgctgatactgccgtgtattactgcgcattcggctactccgactatgaatataattggttcgatccttggggacagggaacattggtaaccgtgtcatccggaggatgtggcggtggaaaagtggccgcatgtaaggagaaagttgctgctttgaaagagaaggtcgccgcacttaaggaaaaggtcgcagccctgaaagag

SEQ ID NO: 29 - protein, 501 aa, synthetic construct
  REGION 1..501: note = First and Third Polypeptide Chains of the Exemplary PD-1 x LAG-3 Fc-DART-2 diabody
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGSGGGGQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 1503 bp, synthetic construct
  misc_feature 1..1503: note = Polynucleotide Encoding the First and Third Polypeptide Chains of the Exemplary PD-1 x LAG-3 Fc-DART-2 diabody
  source 1..1503: mol_type = other DNA
gagatcgtacttacccagtctcccgccaccctttccctgagtcctggtgagcgggccactctttcctgtcgcgcaagccaatcagtttctagctacctcgcatggtatcagcagaagccagggcaggcacccaggcttctcatctatgacgccagtaaccgcgcaaccgggatacctgctagattttccggcagtggatctgggaccgatttcacactgacaatttcatccttggaaccagaagatttcgcagtctactactgccagcaatcttccaactggccaagaactttcggacaggggaccaaagtggaaattaaaggtggcggatccggcggcggaggccaggtccagctccagcaatggggagccgggctgctgaaaccctctgaaacactgagtctcacatgtgccgtttatggaggttccttctccgattattactggaactggattcgtcagcctcccggcaagggcctggagtggatcggtgagattaaccacaatggcaataccaatagcaatcctagtttgaaatctcgcgtcactctttccctcgatacaagcaaaaaccagttttctttgaaattgcgatctgtaactgctgctgatactgccgtgtattactgcgcattcggctactccgactatgaatataattggttcgatccttggggacagggaacattggtaaccgtgtcatccggaggatgtggcggtggagaagtggccgcatgtgagaaagaggttgctgctttggagaaggaggtcgctgcacttgaaaaggaggtcgcagccctggagaaactggagcccaaatctgctgacaaaactcacacatgcccaccgtgcccagcacctgaagccgcggggggaccgtcagtcttcctcttccccccaaaacccaaggacaccctctatatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 31 - protein, 262 aa, synthetic construct
  REGION 1..262: note = Second and Fourth Polypeptide Chains of the Exemplary PD-1 x LAG-3 Fc-DART-2 diabody
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKGGGSGGGGQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGCGGGKVAACKEKVAALKEKVAALKEKVAALKE

SEQ ID NO: 32 - DNA, 786 bp, synthetic construct
  misc_feature 1..786: note = Polynucleotide Encoding the Second and Fourth Polypeptide Chains of the Exemplary PD-1 x LAG-3 Fc-DART-2 diabody
  source 1..786: mol_type = other DNA
gaaattgtcctgacacagtctcccgcaaccctgagtttgagtcctggggagcgagcaactctctcctgccgagcctcccagagtatctcctcctacctcgcctggtaccaacagaagccagggcaggctccaaggctgcttatctatgacgcctctaaccgcgcaactgggattcccgcacgcttctccggctctggttccggcacagactttacacttactatctctagcctggagccagaagactttgccgtgtactattgtcagcaacgttccaattggccccttacctttgggcagggcactaacttggaaatcaaaggtggcggatccggcggcggaggccaggttcagctggtcgagagtggtggcggcgttgtgcaacctgggcgttccctccgattggactgtaaagcttccggcattactttctcaaattccggcatgcattgggtgaggcaagcccctggaaaagggctcgaatgggtggctgtgatttggtacgatggcagcaaacggtactacgccgattctgttaagggccgctttaccatctcccgcgataactcaaagaacacactgtttctgcaaatgaatagtcttagagccgaggacaccgccgtgtactactgtgccacaaatgacgattattgggggcagggcacattggtcacagtgtcttccggaggatgtggcggtggaaaagtggccgcatgtaaggagaaagttgctgctttgaaagagaaggtcgccgcacttaaggaaaaggtcgcagccctgaaagag

(19) United States
(12) Patent Application Publication
(10) Pub. No.: US 2025/0304685 A1
(43) Pub. Date: Oct. 2, 2025

(54) COVALENTLY BONDED DIABODIES HAVING IMMUNOREACTIVITY WITH PD-1 AND LAG-3, AND METHODS OF USE THEREOF

(71) Applicant: MacroGenics, Inc., Rockville, MD (US)

(72) Inventors: Ezio BONVINI, Potomac, MD (US); Leslie S. JOHNSON, Rockville, MD (US); Kalpana SHAH, Boyds, MD (US); Ross LA MOTTE-MOHS, Boyds, MD (US); Paul A. MOORE, North Potomac, MD (US); Scott KOENIG, Rockville, MD (US)

(21) Appl. No.: 19/239,433

(22) Filed: Jun. 16, 2025

Related U.S. Application Data
(63) Continuation of application No. 17/375,821, filed on Jul. 14, 2021.

(51) Int. Cl. C07K 16/28; C07K 16/46

(52) U.S. Cl. CPC C07K 16/2803; C07K 16/2818; C07K 16/468; C07K 2317/31; C07K 2317/626; C07K 2317/74

(57) ABSTRACT

The present invention is directed to bi-specific diabodies that comprise two or more polypeptide chains and which possess at least one Epitope-Binding Site that is immunospecific for an epitope of PD-1 and at least one Epitope-Binding Site that is immunospecific for an epitope of LAG-3 (i.e., a “PD-1×LAG-3 bi-specific diabody”). More preferably, the present invention is directed to bi-specific diabodies that comprise four polypeptide chains and which possess two Epitope-Binding Sites that are immunospecific for one (or two) epitope(s) of PD-1 and two Epitope-Binding Site that are immunospecific for one (or two) epitope(s) of LAG-3 (i.e., a “PD-1×LAG-3 bi-specific, tetra-valent diabody”). The present invention also is directed to such diabodies that additionally comprise an immunoglobulin Fc Domain (“bi-specific Fc diabodies and bi-specific, tetra-valent, Fc diabodies”). The diabodies of the present invention are capable of simultaneously binding to PD-1 and to LAG-3, particularly as such molecules are arrayed on the surfaces of human cells. The invention is directed to pharmaceutical compositions that contain such diabodies, and to methods involving the use of such diabodies in the treatment of cancer and other diseases and conditions.

### CROSS-REFERENCE TO RELATED APPLICATIONS

This patent application is a continuation of U.S. patent application Ser. No. 17/375,821, filed Jul. 14, 2021, which is a continuation of U.S. patent application Ser. No. 16/189,071, filed Nov. 13, 2018, which is a continuation of U.S. patent application Ser. No. 15/321,279, filed Dec. 22, 2016, which is a national stage of International Patent Application No. PCT/US2015/036634, filed Jun. 19, 2015, which claims the benefit of U.S. Provisional Application No. 62/017,467, filed Jun. 26, 2014, each of which applications is incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML copy, created on Jun. 16, 2025, is named 0260-0022_SL_ST26.xml and is 49 KB in size.

### BACKGROUND OF THE INVENTION

### Field of the Invention

The present invention is directed to bi-specific diabodies that comprise two or more polypeptide chains and which possess at least one Epitope-Binding Site that is immunospecific for an epitope of PD-1 and at least one Epitope-Binding Site that is immunospecific for an cpitope of LAG-3 (i.e., a “PD-1×LAG-3 bi-specific diabody”). More preferably, the present invention is directed to bi-specific diabodies that comprise four polypeptide chains and which possess two Epitope-Binding Sites that are immunospecific for one (or two) epitopc(s) of PD-1 and two Epitope-Binding Site that are immunospecific for one (or two) cpitope(s) of LAG-3 (i.e., a “PD-1×LAG-3 bi-specific, tetra-valent diabody”). The present invention also is directed to such diabodies that additionally comprise an immunoglobulin Fc Domain (“bi-specific Fc diabodies” and “bi-specific, tetra-valent, Fc diabodies”). The diabodies of the present invention are capable of simultaneously binding to PD-1 and to LAG-3, particularly as such molecules are arrayed on the surfaces of human cells. The invention is directed to pharmaceutical compositions that contain such diabodies, and to methods involving the use of such diabodies in the treatment of cancer and other diseases and conditions.

### Description of Related Art

### I. Cell-Mediated Immune Responses

The immune system of humans and other mammals is responsible for providing protection against infection and disease. Such protection is provided both by a humoral immune response and by a cell-mediated immune response. The humoral response results in the production of antibodies and other biomolecules that are capable of recognizing and neutralizing foreign targets (antigens). In contrast, the cell-mediated immune response involves the activation of macrophages, Natural Killer cells (NK), and antigen-specific cytotoxic T-lymphocytes by T-cells, and the release of various cytokines in response to the recognition of an antigen (Dong, C. et al. (2003) “Immune Regulation by Novel Costimulatory Molecules,” Immunolog. Res. 28(1):39-48).

The ability of T-cells to optimally mediate an immune response against an antigen requires two distinct signaling interactions (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). First, antigen that has been arrayed on the surface of Antigen-Presenting Cells (APC) must be presented to an antigen-specific naive CD4⁺ T-cell. Such presentation delivers a signal via the T-Cell Receptor (TCR) that directs the T-cell to initiate an immune response that will be specific to the presented antigen. Second, a series of co-stimulatory and inhibitory signals, mediated through interactions between the APC and distinct T-cell surface molecules, triggers first the activation and proliferation of the T-cells and ultimately their inhibition. Thus, the first signal confers specificity to the immune response whereas the second signal serves to determine the nature, magnitude and duration of the response.

The immune system is tightly controlled by costimulatory and co-inhibitory ligands and receptors. These molecules provide the second signal for T-cell activation and provide a balanced network of positive and negative signals to maximize immune responses against infection while limiting immunity to self (Wang, L. et al. (Mar. 7, 2011) “VISTA, A Novel Mouse Ig Superfamily Ligand That Negatively Regulates T-Cell Responses,” J. Exp. Med. 10.1084/jem.20100619:1-16; Lepenies, B. et al. (2008) “The Role Of Negative Costimulators During Parasitic Infections,” Endocrine, Metabolic & Immune Disorders—Drug Targets 8:279-288). Of particular importance is binding between the B7.1 (CD80) and B7.2 (CD86) ligands of the Antigen-Presenting Cell and the CD28 and CTLA-4 receptors of the CD4⁺ T-lymphocyte (Sharpe, A. H. et al. (2002) “The B7-CD28 Superfamily,” Nature Rev. Immunol. 2:116-126; Dong, C. et al. (2003) “Immune Regulation by Novel Costimulatory Molecules,” Immunolog. Res. 28(1):39-48; Lindley, P.S. et al. (2009) “The Clinical Utility Of Inhibiting CD28-Mediated Costimulation,” Immunol. Rev. 229:307-321). Binding of B7.1 or of B7.2 to CD28 stimulates T-cell activation; binding of B7.1 or B7.2 to CTLA-4 inhibits such activation (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321; Greenwald, R. J. et al. (2005) “The B7 Family Revisited,” Ann. Rev. Immunol. 23:515-548). CD28 is constitutively expressed on the surface of T-cells (Gross, J., et al. (1992) “Identification And Distribution Of The Costimulatory Receptor CD28 In The Mouse,” J. Immunol. 149:380-388), whereas CTLA4 expression is rapidly up-regulated following T cell activation (Linsley, P. et al. (1996) “Intracellular Trafficking Of CTLA4 And Focal Localization Towards Sites Of TCR Engagement,” Immunity 4:535-543). Since CTLA4 is the higher affinity receptor (Sharpe, A.H. et al. (2002) “The B7-CD28 Superfamily,” Nature Rev. Immunol. 2:116-126), binding first initiates T-cell proliferation (via CD28) and then inhibits it (via nascent expression of CTLA4), thereby dampening the effect when proliferation is no longer needed.

Further investigations into the ligands of the CD28 receptor have led to the identification and characterization of a set of related B7 molecules (the “B7 Superfamily”) (Coyle, A. J. et al. (2001) “The Expanding B7 Superfamily: Increasing Complexity In Costimulatory Signals Regulating T-Cell Function,” Nature Immunol. 2 (3): 203-209; Sharpe, A. H. et al. (2002) “The B7-CD28 Superfamily,” Nature Rev. Immunol. 2:116-126; Greenwald, R. J. et al. (2005) “The B7 Family Revisited,” Ann. Rev. Immunol. 23:515-548; Collins, M. et al. (2005) “The B7 Family Of Immune-Regulatory Ligands,” Genome Biol. 6:223.1-223.7; Loke, P. et al. (2004) “Emerging Mechanisms Of Immune Regulation: The Extended B7 Family And Regulatory T-Cells.” Arthritis Res. Ther. 6:208-214; Korman, A. J. et al. (2007) “Checkpoint Blockade in Cancer Immunotherapy,” Adv. Immunol. 90:297-339; Flies, D. B. et al. (2007) “The New B7s: Playing a Pivotal Role in Tumor Immunity,” J. Immunother. 30(3):251-260; Agarwal, A. et al. (2008) “The Role Of Positive Costimulatory Molecules In Transplantation And Tolerance,” Curr. Opin. Organ Transplant. 13:366-372; Lenschow, D. J. et al. (1996) “CD28/B7 System of T-Cell Costimulation,” Ann. Rev. Immunol. 14:233-258; Wang, S. et al. (2004) “Co-Signaling Molecules Of The B7-CD28 Family In Positive And Negative Regulation Of T Lymphocyte Responses,” Microbes Infect. 6:759-766). There are currently several known members of the family: B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins, M. et al. (2005) “The B7 Family Of Immune-Regulatory Ligands,” Genome Biol. 6:223.1-223.7; Flajnik, M. F. et al. (2012) “Evolution Of The B7 Family: Co-Evolution Of B7H6 And Nkp30, Identification Of A New B7 Family Member, B7H7, And Of B7′s Historical Relationship With The MHC,” Immunogenetics epub doi.org/10.1007/s00251-012-0616-2).

### II. PD-1

Programmed Death-1 (“PD-1”) is an approximately 31 kD type I membrane protein member of the extended CD28/CTLA4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895; United States Patent Application Publication No. 2007/0202100; 2008/0311117; 2009/00110667; U.S. Pat. Nos. 6,808,710; 7,101,550; 7,488,802; 7,635,757; 7,722,868; PCT Publication No. WO 01/14557). Compared to CTLA4, PD-1 more.

PD-1 is expressed on activated T-cells, B cells, and monocytes (Agata, Y. et al. (1996) “Expression Of The PD-I Antigen On The Surface Of Stimulated Mouse T And B Lymphocytes,” Int. Immunol. 8(5):765-772; Yamazaki, T. et al. (2002) “Expression Of Programmed Death 1 Ligands By Murine T-Cells And APC,” J. Immunol. 169:5538-5545) and at low levels in Natural Killer (NK) T-cells (Nishimura, H. et al. (2000) “Facilitation Of Beta Selection And Modification Of Positive Selection In The Thymus Of PD-1-Deficient Mice,” J. Exp. Med. 191:891-898; Martin-Orozco, N. et al. (2007) “Inhibitory Costimulation And Anti-Tumor Immunity,” Semin. Cancer Biol. 17(4):288-298).

The extracellular region of PD-1 consists of a single immunoglobulin (Ig)V domain with 23% identity to the equivalent domain in CTLA4 (Martin-Orozco, N. et al. (2007) “Inhibitory Costimulation And Anti-Tumor Immunity,” Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y. et al. (1992) “Induced Expression Of PD-1, A Novel Member Of The Immunoglobulin Gene Superfamily, Upon Programmed Cell Death,” EMBO J. 11:3887-3895; Blank, C. et al. (Epub 2006 Dec. 29) “Contribution Of The PD-L1/PD-1 Pathway To T-Cell Exhaustion: An Update On Implications For Chronic Infections And Tumor Evasion Cancer,” Immunol. Immunother. 56(5):739-745).

PD-1 mediates its inhibition of the immune system by binding to B7-H1 and B7-DC (Flies, D. B. et al. (2007) “The New B7s: Playing a Pivotal Role in Tumor Immunity,” J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication No. WO 01/39722; WO 02/086083).

B7-H1 and B7-DC are binding ligands that are broadly expressed on the surfaces of human and murine tissues, such as heart, placenta, muscle, fetal liver, spleen, lymph nodes, and thymus as well as murine liver, lung, kidney, islets cells of the pancreas and small intestine (Martin-Orozco, N. et al. (2007) “Inhibitory Costimulation And Anti-Tumor Immunity,” Semin. Cancer Biol. 17(4):288-298). In humans, B7-H1 protein expression has been found in human endothelial cells (Chen, Y. et al. (2005) “Expression of B7-H1 in Inflammatory Renal Tubular Epithelial Cells,” Nephron. Exp. Nephrol. 102:e81-c92; de Haij, S. et al. (2005) “Renal Tubular Epithelial Cells Modulate T-Cell Responses Via ICOS-L And B7-H1” Kidney Int. 68:2091-2102; Mazanet, M. M. et al. (2002) “B7-H1 Is Expressed By Human Endothelial Cells And Suppresses T-Cell Cytokine Synthesis,” J. Immunol. 169:3581-3588), myocardium (Brown, J. A. et al. (2003) “Blockade Of Programmed Death-1 Ligands On Dendritic Cells Enhances T-Cell Activation And Cytokine Production,” J. Immunol. 170:1257-1266), syncyciotrophoblasts (Petroff, M. G. et al. (2002) “B7 Family Molecules: Novel Immunomodulators At The Maternal-Fetal Interface,” Placenta 23:S95-S101). The molecules are also expressed by resident macrophages of some tissues, by macrophages that have been activated with interferon (IFN)-γ or tumor necrosis factor (TNF)-α (Latchman, Y. et al. (2001) “PD-L2 Is A Second Ligand For PD-1 And Inhibits T-Cell Activation,” Nat. Immunol 2:261-268), and in tumors (Dong, H. (2003) “B7-H1 Pathway And Its Role In The Evasion Of Tumor Immunity,” J. Mol. Med. 81:281-287).

The interaction between B7-H1 and PD-1 has been found to provide a crucial negative co-stimulatory signal to T and B cells (Martin-Orozco, N. et al. (2007) “Inhibitory Costimulation And Anti-Tumor Immunity,” Semin. Cancer Biol. 17(4):288-298) and functions as a cell death inducer (Ishida, Y. et al. (1992) “Induced Expression Of PD-1, A Novel Member Of The Immunoglobulin Gene Superfamily, Upon Programmed Cell Death,” EMBO J. 11:3887-3895; Subudhi, S. K. et al. (2005) “The Balance Of Immune Responses: Costimulation Verse Coinhibition,” J. Molec. Med. 83:193-202). More specifically, interaction between low concentrations of the PD-1 receptor and the B7-Hl ligand has been found to result in the transmission of an inhibitory signal that strongly inhibits the proliferation of antigen-specific CD8⁺ T-cells; at higher concentrations the interactions with PD-1 do not inhibit T cell proliferation but markedly reduce the production of multiple cytokines (Sharpe, A. H. et al. (2002) “The B7-CD28 Superfamily,” Nature Rev. Immunol. 2:116-126). T cell proliferation and cytokine production by both resting and previously activated CD4 and CD8 T-cells, and even naive T-cells from umbilical-cord blood, have been found to be inhibited by soluble B7-H1-Fc fusion proteins (Freeman, G. J. et al. (2000) “Engagement Of The PD-1 Immunoinhibitory Receptor By A Novel B7 Family Member Leads To Negative Regulation Of Lymphocyte Activation,” J. Exp. Med. 192:1-9; Latchman, Y. et al. (2001) “PD-L2 Is A Second Ligand For PD-1 And Inhibits T-Cell Activation,” Nature Immunol. 2:261-268; Carter, L. et al. (2002) “PD-1: PD-L inhibitory pathway affects both CD4(+) and CD8(+) T-cells and is overcome by IL-2,” Eur. J. Immunol. 32(3):634-643; Sharpe, A. H. et al. (2002) “The B7-CD28 Superfamily,” Nature Rev. Immunol. 2:116-126).

The role of B7-H1 and PD-1 in inhibiting T-cell activation and proliferation has suggested that these biomolecules might serve as therapeutic targets for treatments of inflammation and cancer. Thus, the use of anti-PD-1 antibodies to treat infections and tumors and up-modulate an adaptive immune response has been proposed (sec, United States Patent Application Publication Nos. 2010/0040614; 2010/0028330; 2004/0241745; 2008/0311117; 2009/0217401; U.S. Pat. Nos. 7,521,051; 7,563,869; 7,595,048; PCT Publications Nos. WO 2004/056875; WO 2008/083174). Antibodies capable of immunospecifically binding to PD-1 have been reported by Agata, T. et al. (1996) “Expression Of The PD-1 Antigen On The Surface Of Stimulated Mouse T And B Lymphocytes,” Int. Immunol. 8(5):765-772 and Berger, R. et al. (2008) “Phase I Safety And Pharmacokinetic Study Of CT-011, A Humanized Antibody Interacting With PD-1, In Patients With Advanced Hematologic Malignancies,” Clin. Cancer Res. 14(10):3044-3051 (sec, also, U.S. Pat. Nos. 8,008,449 and 8,552,154; US Patent Publications No. 2007/0166281; 2012/0114648; 2012/0114649; 2013/0017199; 2013/0230514 and 2014/0044738; and PCT Patent Publications WO 2003/099196; WO 2004/004771; WO 2004/056875; WO 2004/072286; WO 2006/121168; WO 2007/005874; WO 2008/083174; WO 2009/014708; WO 2009/073533; WO 2012/135408, WO 2012/145549 and WO 2013/014668).

### III. LAG-3

Lymphocyte activation gene 3 (LAG-3, CD223) is a cell-surface receptor protein that is expressed by activated CD4⁺ and CD8⁺ T-cells and NK cells, and is constitutively expressed by plasmacytoid dendritic cells; LAG-3 is not expressed by B cells, monocytes or any other cell types tested (Workman, C. J. et al. (2009) “LAG-3 Regulates Plasmacytoid Dendritic Cell Homeostasis,” J. Immunol. 182 (4): 1885-1891).

LAG-3 has been found to be closely related to the T-cell co-receptor CD4 (Grosso, J. F. et al. (2009) “Functionally Distinct LAG-3 and PD-1 Subsets on Activated and Chronically Stimulated CD8 T-Cells,” J. Immunol. 182(11):6659-6669; Huang, C. T. et al. (2004) “Role Of LAG-3 In Regulatory T-Cells,” Immunity 21:503-513; Workman, C. J. et al. (2009) “LAG-3 Regulates Plasmacytoid Dendritic Cell Homeostasis,” J. Immunol. 182(4):1885-1891). Like CD4, LAG-3 also binds to MHC class II molecules but does so with significantly higher affinity (Workman, C. J. et al. (2002) “Phenotypic Analysis Of The Murine CD4-Related Glycoprotein, CD223 (LAG-3),” Eur. J. Immunol. 32:2255-2263; Huard, B. et al. (1995) “CD4/l Major Histocompatibility Complex Class Il Interaction Analyzed With CD4-And Lymphocyte Activation Gene-3 (LAG-3)-Ig Fusion Proteins,” Eur. J. Immunol. 25:2718-2721; Huard, B. et al. (1994) “Cellular Expression And Tissue Distribution Of The Human LAG-3-Encoded Protein, An MHC Class II Ligand,” Immunogenetics 39:213-217).

Studies have shown that LAG-3 plays an important role in negatively regulating T-cell proliferation, function and homeostasis (Workman, C.J. et al. (2009) “LAG-3 Regulates Plasmacytoid Dendritic Cell Homeostasis,” J. Immunol. 182 (4): 1885-1891; Workman, C. J. et al. (2002) “Cutting Edge: Molecular Analysis Of The Negative Regulatory Function Of Lymphocyte Activation Gene-3,” J. Immunol. 169:5392-5395; Workman, C. J. et al. (2003) “The CD4-Related Molecule, LAG-3 (CD223), Regulates The Expansion Of Activated T-Cells,” Eur. J. Immunol. 33:970-979; Workman, C. J. (2005) “Negative Regulation Of T-Cell Homeostasis By Lymphocyte Activation Gene-3 (CD223),” J. Immunol. 174:688-695; Hannier, S. et al. (1998) “CD3/TCR Complex-Associated Lymphocyte Activation Gene-3 Molecules Inhibit CD3/TCR Signaling,” J. Immunol. 161:4058-4065; Huard, B. et al. (1994) “Lymphocyte-Activation Gene 3/Major Histocompatibility Complex Class II Interaction Modulates The Antigenic Response Of CD4⁺ T Lymphocytes,” Eur. J. Immunol. 24:3216-3221).

Studies have suggested that inhibiting LAG-3 function through antibody blockade can reverse LAG-3-mediated immune system inhibition and partially restore effector function (Grosso, J. F. et al. (2009) “Functionally Distinct LAG-3 and PD-1 Subsets on Activated and Chronically Stimulated CD8 T-Cells,” J. Immunol. 182(11):6659-6669; Grosso, J. F. et al. (2007) “LAG-3 Regulates CD8⁺ T-Cell Accumulation And Effector Function During Self And Tumor Tolerance,” J. Clin. Invest. 117:3383-3392). LAG-3 has been found to negatively regulate T cell expansion via inhibition of T Cell Receptor (TCR)-induced calcium fluxes, and controls the size of the memory T cell pool (Matsuzaki, J. et al. (2010) “Tumor-Infiltrating NY-ESO-1-Specific CD8+ T-Cells Are Negatively Regulated By LAG-3 And PD-1 In Human Ovarian Cancer,” Proc. Natl. Acad. Sci. (U.S.A.) 107(17):7875-7880; Workman C. J., et al. (2004) “Lymphocyte Activation Gene-3 (CD223) Regulates The Size Of The Expanding T-Cell Population Following Antigen Activation in vivo,” J. Immunol. 172:5450-5455).

Despite prior advances, a need remains for improved compositions capable of more vigorously directing the body's immune system to attack cancer cells or pathogen-infected cells, especially at lower therapeutic concentrations. For although the adaptive immune system can be a potent defense mechanism against cancer and disease, it is often hampered by immune suppressive mechanisms in the tumor microenvironment, such as the expression of PD-1 and LAG-3. Coinhibitory molecules expressed by tumor cells, immune cells, and stromal cells in the tumor milieu can dominantly attenuate T-cell responses against cancer cells.

As described in detail below, the present invention addresses this need by providing PD-1×LAG-3 bi-specific, tetra-valent, diabodies. Such diabodies are capable of binding to PD-1 and LAG-3 cell-surface molecules that are present on the surfaces of exhausted and tolerant tumor-infiltrating lymphocytes, and of thereby impairing the ability of such cell-surface molecules to bind to their receptor ligands. As such, the PD-1×LAG-3 bi-specific diabodies of the present invention act to block PD-1 and LAG-3-mediated immune system inhibition, and thereby promote the continued activation of the immune system. This attribute permits such bi-specific diabodies to have utility in the treatment of cancer and pathogen-associated diseases and conditions. The invention is directed to such diabodies and to methods for their use.

### BRIEF DESCRIPTION OF THE FIGURES:

FIG. 1 shows a diagrammatic representation of the Domains of a basic DART®.

FIG. 2 shows a diagrammatic representation of the Domains of an Fc-bearing DART®.

FIG. 3 shows a diagrammatic representation of the Domains of an Fc-DART®.

FIG. 4 shows a diagrammatic representation of the Domains of a preferred PD-1×LAG-3 bi-specific, tetra-valent, diabody of the present invention. Four polypeptide chains, two of which have the Domains of polypeptide chains 1 and 3, and two of which have the Domains of polypeptide chains 2 and 4, complex together to form the diabody. Disulfide bonds (shown as striped lines) covalently link polypeptide chains 1 and 2, polypeptide chains 1 and 3 and polypeptide chains 3 and 4. The Variable Light Chain Domains and Variable Heavy Chain Domains of the same polypeptide chain are directed to different epitopes (either PD-1 or LAG-3), such that the resulting diabody has two Epitope-Binding Domains that are immunospecific for PD-1 and two Epitope-Binding Domains that are immunospecific for LAG-3.

FIG. 5 shows a diagram of the protocol for assessing the ability of anti-PD-1 and anti-LAG-3 antibodies to enhance the proliferation of T-cells.

FIG. 6 shows that the addition of PD-1 mAb 1 (5C4; BMS-936558), PD-1 mAb 2 (MK-3475; Merck, lambrolizumab) and PD-1 mAb 3 (EH12.2H7; Dana Farber) at the start of the allo-MLR assay, induced a strong T-cell proliferation response compared to IgG1 isotype control antibody. Also shown are the proliferative responses obtained with PD-1 mAb 4 (CT-011; CureTech, BAT-1), an anti-CTLA mAb and LAG-3 mAb 1 (25F7; BMS-986016, Medarex/BMS). Responder (R) cells are pan T cells; stimulator (S)cells are mature dendritic cells (mDCs).

FIG. 7 shows the results of an evaluation of LAG-3 mAb 1 (25F7; BMS-986016, Medarex/BMS), for T-cell proliferative potential either alone or in combination with PD-1mAb 1 (5C4; BMS-936558)), PD-1 mAb 2 (MK-3475; Merck, lambrolizumab). Responder (R) cells are pan T cells; stimulator (S)cells are mature dendritic cells (mDCs).

FIG. 8 shows that soluble human LAG-3 (shLAG-3), which binds to human HLA-class II molecules expressed on both APCs and CD4 T-cells, induced a robust proliferative response compared to IgG isotype or responder (R) (pan T cells) plus stimulator (S) (mature dendritic cells (mDCs) control wells.

FIGS. 9A-9B show that the PD-1×LAG-3 bi-specific diabodies of the present invention induced potent T-cell proliferative responses when compared against anti-PD-1 mAb (5C4) or anti-LAG-3 mAb (25F7). FIG. 9A shows the T-cell proliferative responses obtained using the preferred PD-1×LAG-3 bi-specific, tetra-valent diabodies of the present invention (PD-1×LAG-Fc-DART®-1 and PD-1×LAG-Fc-DART®-2), PD-1 mAb 1 (5C4; BMS-936558)), LAG-3 mAb 1 (25F7; BMS-986016, Medarex/BMS), soluble human LAG-3 (ShLAG-3), a control IgG, responder+stimulator cells (pan T cells and mature dendritic cells; R+S) and stimulator cells (mature dendritic cells; S). FIG. 9B shows the same data for PD-1×LAG-Fc-DART®-1 and PD-1×LAG-Fc-DART®-2, PD-1 mAb 1 (5C4; BMS-936558)), LAG-3 mAb 1 (25F7; BMS-986016, Medarex/BMS), and PD-1 mAb 1 (5C4; BMS-936558))+LAG-3 mAb 1 (25F7; BMS-986016, Medarex/BMS), using a different y-axis scale.

### SUMMARY OF THE INVENTION

The present invention is directed to bi-specific diabodies that comprise two or more polypeptide chains and which possess at least one Epitope-Binding Site that is immunospecific for an epitope of PD-1 and at least one Epitope-Binding Site that is immunospecific for an epitope of LAG-3 (i.e., a “PD-1×LAG-3 bi-specific diabody”). More preferably, the present invention is directed to bi-specific diabodies that comprise four polypeptide chains and which possess two Epitope-Binding Sites that are immunospecific for one (or two) epitopc(s) of PD-1 and two Epitope-Binding Site that are immunospecific for one (or two) epitope(s) of LAG-3 (i.e., a “PD-1×LAG-3 bi-specific, tetra-valent diabody”). The present invention also is directed to such diabodies that additionally comprise an immunoglobulin Fc Domain (“bi-specific Fc diabodies” and “bi-specific, tetra-valent, Fc diabodies”). The diabodies of the present invention are capable of simultaneously binding to PD-1 and to LAG-3, particularly as such molecules are arrayed on the surfaces of human cells. The invention is directed to pharmaceutical compositions that contain such diabodies, and to methods involving the use of such diabodies in the treatment of cancer and other diseases and conditions.

In detail, the invention provides a bi-specific Fc diabody capable of immunospecific binding to an epitope of PD-1 and to an epitope of LAG-3, wherein the diabody comprises four polypeptide chains, each having an amino terminus and a carboxy terminus, and wherein:
(A) the first and second polypeptide chains are covalently bonded to one another, the second and third polypeptide chains are covalently bonded to one another, and the third and fourth polypeptide chains are covalently bonded to one another;
(B) the first and third polypeptide chains of the diabody each comprise, in the N-terminal to C-terminal direction, a Light Chain Variable Domain of an antibody that is immunospecific for PD-1 or LAG-3, a Heavy Chain Variable Domain of an antibody that is immunospecific for LAG-3 or PD-1, a Heterodimer-Promoting Domain and a CH2-CH3 Domain, wherein the Light Chain Variable Domains and the Heavy Chain Variable Domains are incapable of associating to form an Epitope-Binding Site capable of binding an epitope of PD-1 or an epitope of LAG-3; and
(C) the second and the fourth polypeptide chains of the diabody each comprise, in the N-terminal to C-terminal direction, a Light Chain Variable Domain of an antibody that is immunospecific for PD-1 or LAG-3, a Heavy Chain Variable Domain of an antibody that is immunospecific for LAG-3 or PD-1, and a Heterodimer-Promoting Domain, wherein the Light Chain Variable Domains and the Heavy Chain Variable Domains are incapable of associating to form an Epitope-Binding Site capable of binding an epitope of PD-1 or an epitope of LAG-3;
   and wherein:
(I) (1) the Light Chain Variable Domain of the first polypeptide chain and the Heavy Chain Variable Domain of the second polypeptide chain associate to form a first Epitope-Binding Site and the Heavy Chain Variable Domain of the first polypeptide chain and the Light Chain Variable Domain of the second polypeptide chain associate to form a second Epitope-Binding Site; and
   (2) the Light Chain Variable Domain of the third polypeptide chain and the Heavy Chain Variable Domain of the fourth polypeptide chain associate to form a third Epitope-Binding Site and the Heavy Chain Variable Domain of the third polypeptide chain and the Light Chain Variable Domain of the fourth polypeptide chain associate to form a fourth Epitope-Binding Site;
   wherein two of the formed Epitope-Binding Sites are capable of immunospecifically binding to an epitope of PD-1 and two of the formed Epitope-Binding Sites are capable of immunospecifically binding to an epitope of LAG-3;
II. the Heterodimer-Promoting Domains of the first and third polypeptide chains differ and have an amino acid sequence selected from the group consisting of: SEQ ID NO:16 and SEQ ID NO:17; and
III. the CH2-CH3 Domains of the first and third polypeptide chains associate to form an Fc Domain.

The invention also concerns the embodiment of such a bi-specific Fc diabody wherein the CH2-CH3 Domains of the first and third polypeptide chains each have the amino acid sequence of SEQ ID NO:24.

The invention also concerns the embodiment of such bi-specific Fc diabodies wherein the Heavy Chain Variable Domain of an antibody that is immunospecific for LAG-3 has the amino acid sequence of SEQ ID NO:11, and wherein the Light Chain Variable Domain of an antibody that is immunospecific for LAG-3 has the amino acid sequence of SEQ ID NO:12.

The invention also concerns the embodiment of such bi-specific Fc diabodies wherein the Heavy Chain Variable Domain of an antibody that is immunospecific for PD-1 has the amino acid sequence of SEQ ID NO:2, and wherein the Light Chain Variable Domain of an antibody that is immunospecific for PD-1 has the amino acid sequence of SEQ ID NO:3.

The invention also concerns a pharmaceutical composition that comprises an effective amount of any of the above-indicated Fc diabodies, and a pharmaceutically acceptable carrier.

The invention also concerns the embodiment of such a pharmaceutical composition wherein the effective amount of the bi-specific Fc diabody is an amount effective to treat cancer in a recipient individual in need of such treatment.

The invention also concerns the embodiment of such pharmaceutical compositions wherein the cancer is an adrenal gland cancer, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, or a uterine cancer.

The invention also concerns the embodiment of such pharmaceutical compositions wherein the effective amount of the bi-specific Fc diabody is an amount effective to treat a disease associated with the presence of a pathogen in a recipient individual in need of such treatment.

The invention also concerns the embodiment of such a pharmaceutical composition wherein the pathogen is a bacterium a fungus or a virus.

The invention also concerns a method of treating cancer which comprises administering an effective amount of such pharmaceutical compositions to an individual in need thereof.

The invention also concerns a method of treating a disease associated with the presence of a pathogen which comprises administering an effective amount of the pharmaceutical composition of any of claims 8-9 to an individual in need thereof.

### DETAILED DESCRIPTION OF THE INVENTION

The present invention is directed to bi-specific diabodies that comprise two or more polypeptide chains and which possess at least one Epitope-Binding Site that is immunospecific for an epitope of PD-1 and at least one Epitope-Binding Site that is immunospecific for an epitope of LAG-3 (i.e., a “PD-1×LAG-3 bi-specific diabody”). More preferably, the present invention is directed to bi-specific diabodies that comprise four polypeptide chains and which possess two Epitope-Binding Sites that are immunospecific for one (or two) epitope(s) of PD-1 and two Epitope-Binding Site that are immunospecific for one (or two) epitope(s) of LAG-3 (i.e., a “PD-1×LAG-3 bi-specific, tetra-valent diabody”). The present invention also is directed to such diabodies that additionally comprise an immunoglobulin Fc Domain (“bi-specific Fc diabodies” and “bi-specific, tetra-valent, Fc diabodies”). The diabodies of the present invention are capable of simultaneously binding to PD-1 and to LAG-3, particularly as such molecules are arrayed on the surfaces of human cells. The invention is directed to pharmaceutical compositions that contain such diabodies, and to methods involving the use of such diabodies in the treatment of cancer and other diseases and conditions.

The bi-specific diabodies of the present invention are capable of simultaneously binding to PD-1 and to LAG-3, particularly as such molecules are arrayed on the surfaces of human cells. The invention is directed to pharmaceutical compositions that contain such diabodies, and to methods involving the use of such diabodies in the treatment of cancer and other diseases and conditions. In particular, the PD-1×LAG-3 bi-specific diabodies of the present invention comprise polypeptide chains that are covalently complexed together.

As discussed above, T-cell activation requires two distinct signals. The first signal is provided by the T-Cell Receptor (TCR) expressed on the surface of a T-cell that has recognized a peptide antigen within the context of human leukocyte antigens (HLA) expressed on an Antigen-Presenting Cell (APCs). The second signal is provided by the interaction of cognate pairs of co-stimulatory ligands: B7-1 and B7-2 expressed on APCs and their corresponding receptors: CD28 and CTLA-4 expressed on T-cells.

Within this receptor-ligand axis, engagement of B7-co-stimulator molecules with the CD28 receptor can stimulate T-cell proliferation and subsequently induce the expression of CTLA-4, a negative-regulator and counter-receptor to CD28 that strongly competes for B7-1 and B7-2 ligands so as to “wind down” T-cell activation and proliferative responses. Agonist antibodies that bind CD28 have been shown to induce T-cell effector function and enhance the generation of tumor eradicating immunity and are co-stimulatory in nature. Conversely, antagonists that block CTLA-4 engagement can prevent T-cells from disengaging their effector function while maintaining sustained proliferation that can lead to autoimmunity.

In parallel with the CTLA-4:B7-1/B7-2 axis, which functions to activate the immune system during normal homeostasis and in the priming phase of an immune response against an antigen, a second receptor-ligand axis functions to inhibit the immune system, thereby serving as a counter-point to CTLA-4 during the effector phase of an immune response. This second axis involves the binding of the programmed cell death-1 protein (PD-1) receptor, expressed on the surface of T-cells, to its corresponding ligands: PD-L1 and PD-L2, expressed on Antigen-Presenting Cells (APCs) and epithelial cells, respectively (Chen L. et al. (2013) “Molecular Mechanisms Of T-Cell Co-Stimulation And Co-Inhibition,” Nature Reviews Immunology 13(4):227-242). In contrast to agonist antibodies that bind to CD28 to stimulate T-cell responses, antibodies that bind to either PD-1 or PD-LI antagonize or block PD-1/PD-L1 engagement are capable of maintaining T-cell responses by preventing the delivery of a negative signal toward T-cell. This augments or maintains T-cell proliferation, cytotoxicity, and cytokine secretion. Taken together agonist antibodies, such as anti-CD28, target positive signal pathways and are therefore co-stimulators, while antagonistic antibodies, such as anti-PD-1, target negative signal pathways and are called checkpoint inhibitors.

Although, CTLA-4 and PD-1 represent the canonical checkpoint inhibitors, there exists a growing family of immune modulating receptor-ligand pairs. Lymphocyte activation gene-3 (LAG-3), discussed above, is an additional checkpoint inhibitor target expressed on T-cells that binds to HLA-class II molecules expressed on APCs. LAG-3 is co-expressed with PD-1 on exhausted and tolerant tumor-infiltrating lymphocytes (“TILs”) (Matsuzaki, J. et al. (2010) “Tumor-Infiltrating NY-ESO-1-Specific CD8+ T-Cells Are Negatively Regulated by LAG-3 and PD-1 in Human Ovarian Cancer,” Proc. Natl. Acad. Sci. (U.S.A.) 107(17):7875-7880; Okazaki, T. et al. (2011) “PD-1 and LAG-3 inhibitor Coreceptors Act Synergistically To Prevent Autoimmunity In Mice,” J. Exp. Med. 208(2):395-407), and LAG-3 expression has been reported on T-regulatory cells implicating a role in both tumor immunology and autoimmunity. Animal models have demonstrated that anti-LAG-3 induces potent tumor cradicating immunity sufficient to slow tumor growth, and when given in combination with anti-PD-1 mAb can even trigger complete tumor regression (Woo, S.R. et al. (2012) “Immune Inhibitory Molecules LAG-3 And PD-1 Synergistically Regulate T-Cell Function To Promote Tumoral Immune Escape,” Cancer Res. 72(4):917-927). Combination therapies involving anti-LAG-3 mAb BMS-986016 are currently under carly-phase clinical investigation either alone or in combination with anti-PD-1 mAb (nivolumab/BMS-936558) (see, Creclan, B. C. (2014) “Update on Immune Checkpoint Inhibitors in Lung Cancer,” Cancer Control 21 (1): 80-89)

The bi-specific diabodies of the present invention are capable of binding to PD-1 and LAG-3 cell-surface molecules that are present on the surfaces of exhausted and tolerant tumor-infiltrating lymphocytes, and of thereby impairing the ability of such cell-surface molecules to bind to their receptor ligands. As such, the PD-1×LAG-3 bi-specific diabodies of the present invention are able to attenuate PD-1 and LAG-3-mediated immune system inhibition, and promote continued immune system activation.

### I. General Techniques and General Definitions

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, MOLECULAR CLONING: A LABORATORY MANUAL, Third Edition (Sambrook et al. Eds., 2001) Cold Spring Harbor Press, Cold Spring Harbor, NY; OLIGONUCLEOTIDE SYNTHESIS: METHODS AND APPLICATIONS (Methods in Molecular Biology), Herdewijn, P., Ed., Humana Press, Totowa, NJ; OLIGONUCLEOTIDE SYNTHESIS (Gait, M. J., Ed., 1984); METHODS IN MOLECULAR BIOLOGY, Humana Press, Totowa, NJ; CELL BIOLOGY: A LABORATORY NOTEBOOK (Cellis, J. E., Ed., 1998) Academic Press, New York, NY; ANIMAL CELL CULTURE (Freshney, R. I., Ed., 1987); INTRODUCTION TO CELL AND TISSUE CULTURE (Mather, J. P. and Roberts, P. E., Eds., 1998) Plenum Press, New York, NY; CELL AND TISSUE CULTURE: LABORATORY PROCEDURES (Doyle, A. et al., Eds., 1993-8) John Wiley and Sons, Hoboken, NJ; METHODS IN ENZYMOLOGY (Academic Press, Inc.) New York, NY; WEIR'S HANDBOOK OF EXPERIMENTAL IMMUNOLOGY (Herzenberg, L. A. et al. Eds. 1997) Wiley-Blackwell Publishers, New York, NY; GENE TRANSFER VECTORS FOR MAMMALIAN CELLS (Miller, J. M. et al. Eds., 1987) Cold Spring Harbor Press, Cold Spring Harbor, NY; CURRENT PROTOCOLS IN MOLECULAR BIOLOGY (Ausubel, F. M. et al., Eds., 1987) Greene Pub. Associates, New York, NY; PCR: THE POLYMERASE CHAIN REACTION, (Mullis, K. et al., Eds., 1994) Birkhäuser, Boston MA; CURRENT PROTOCOLS IN IMMUNOLOGY (Coligan, J. E. et al., eds., 1991) John Wiley and Sons, Hoboken, NJ; SHORT PROTOCOLS IN MOLECULAR BIOLOGY (John Wiley and Sons, 1999) Hoboken, NJ; IMMUNOBIOLOGY 7 (Janeway, C. A. et al. 2007) Garland Science, London, UK; Antibodies (P. Finch, 1997) Stride Publications, Devoran, UK; ANTIBODIES: A PRACTICAL APPROACH (D. Catty., ed., 1989) Oxford University Press, USA, New York NY); MONOCLONAL ANTIBODIES: A PRACTICAL APPROACH (Shepherd, P. et al. Eds., 2000) Oxford University Press, USA, New York NY; USING ANTIBODIES: A LABORATORY MANUAL (Harlow, E. et al. Eds., 1998) Cold Spring Harbor Laboratory Press, Cold Spring Harbor, NY; THE ANTIBODIES (Zanetti, M. et al. Eds. 1995) Harwood Academic Publishers, London, UK); and DEVITA, HELLMAN, AND ROSENBERG'S CANCER: PRINCIPLES & PRACTICE OF ONCOLOGY, EIGHTH EDITION, DeVita, V. et al. Eds. 2008, Lippincott Williams & Wilkins, Philadelphia, PA.

As used herein, “antibodies” are immunoglobulin molecules capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also mutants thereof, naturally occurring variants, fusion proteins comprising an antibody portion with an antigen recognition site of the required specificity, humanized antibodies, and chimeric antibodies, and any other modified configuration of the immunoglobulin molecule that comprises an antigen recognition site of the required specificity. Naturally occurring antibodies typically comprise two copies of a “heavy” (“H”) polypeptide chain and two copies of a “light” (“L”) polypeptide chain. Each light chain is comprised of a Light Chain Variable Region (“VL”) and a light chain constant region (“CL”), Each heavy chain is comprised of a Heavy Chain Variable Region (“VH”) and a heavy chain constant region, usually comprised of three domains (CH1, CH2 and CH3). The CH2 and CH3 Domains of the heavy chain polypeptides interact with one another to form an Fc region that is capable of binding to Fc receptors present on the surfaces of immune system cells.

The ability of an intact, unmodified antibody (e.g., an IgG) to bind an epitope of an antigen depends upon the presence of variable Domains on the immunoglobulin light and heavy chains (i.e., the VL and VH Domains, respectively). Interaction of an antibody light chain and an antibody heavy chain and, in particular, interaction of its VL and VH Domains forms one of the epitope-binding sites of the antibody. In contrast, an “scFv” fragment of an antibody comprises a VL and VH Domain of an antibody contained in a single polypeptide chain wherein the Domains are separated by a flexible linker of sufficient length to allow self-assembly of the two Domains into a functional Epitope-Binding Site.

Where self-assembly of the VL and VH Domains is rendered impossible due to a linker of insufficient length (less than about 12 amino acid residues), two of the scFv constructs interact with one another other to form a “diabody,” which is a bi-valent molecule in which the VL of one chain associates with the VH of the other (reviewed in Marvin et al. (2005) “Recombinant Approaches To IgG-Like Bispecific Antibodies,” Acta Pharmacol. Sin. 26:649-658).

In addition to their known uses in diagnostics, antibodies have been shown to be useful as therapeutic agents. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Nearly 200 antibody-based drugs have been approved for use or are under development.

The term “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term monoclonal antibody encompasses not only intact monoclonal antibodies and full-length monoclonal antibodies, but also fragments thereof (such as Fab, Fab', F (ab') 2 Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof.

Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi. In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the bi-specific molecules of the invention as well as a chimeric antibody, a humanized antibody, or a caninized antibody, to improve the affinity, or other characteristics of the antibody. The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

Natural antibodies are capable of binding to only one epitope species (i.e., they are mono-specific), although they can bind multiple copies of that species (i.e., exhibiting bi-valency or multi-valency). A wide variety of recombinant bi-specific antibody formats have been developed (see, e.g., PCT Publication Nos. WO 2008/003116, WO 2009/132876, WO 2008/003103, WO 2007/146968), most of which use linker peptides either to fuse the antibody core (IgA, IgD, IgE, IgG or IgM) to a further binding protein (e.g. scFv) or to fuse e.g. two Fab fragments or scFv. Typically, such approaches involve compromises and trade-offs. For example, PCT Publications Nos. WO 2013/174873, WO 2011/133886 and WO 2010/136172 disclose that the use of linkers may cause problems in therapeutic settings, and teaches a tri-specific antibody in which the CL and CHI Domains are switched from their respective natural positions and the VL and VH Domains have been diversified (WO 2008/027236; WO 2010/108127) to allow them to bind to more than one antigen. Thus, the molecules disclosed in these documents trade binding specificity for the ability to bind additional antigen species. PCT Publications Nos. WO 2013/163427 and WO 2013/119903 disclose modifying the CH2 Domain to contain a fusion protein adduct comprising a binding domain. The document notes that the CH2 Domain likely plays only a minimal role in mediating effector function. PCT Publications Nos. WO 2010/028797, WO2010028796 and WO 2010/028795 disclose recombinant antibodies whose Fc Regions have been replaced with additional VL and VH Domains, so as to form tri-valent binding molecules. PCT Publications Nos. WO 2003/025018 and WO2003012069 disclose recombinant diabodies whose individual chains contain scFv domains. PCT Publications No. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publications Nos. WO 2014/022540, WO 2013/003652, WO 2012/162583, WO 2012/156430, WO 2011/086091, WO 2007/075270, WO 1998/002463, WO 1992/022583 and WO 1991/003493 disclose adding additional Binding Domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another). Thus, the molecules disclosed in these documents trade native antibody structure for the ability to bind additional antigen species.

The art has additionally noted the ability to produce diabodies that differ from natural antibodies in being capable of binding two or more different epitope species (i.e., exhibiting bi-specificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76 (8): 992; Takemura, S. et al. (2000) “Construction Of A Diabody (Small Recombinant Bispecific Antibody) Using A Refolding System,” Protein Eng. 13(8):583-588; Bacuerle, P. A. et al. (2009) “Bispecific T-Cell Engaging Antibodies For Cancer Therapy,” Cancer Res. 69 (12): 4941-4944).

The design of a diabody is based on the single-chain variable region fragments (scFv). Such molecules are made by linking light and/or heavy chain variable regions by using a short linking peptide. Bird et al. (1988) (“Single-Chain Antigen-Binding Proteins,” Science 242:423-426) describes example of linking peptides which bridge approximately 3.5 nm between the carboxy terminus of one variable region and the amino terminus of the other variable region. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

U.S. Pat. No. 7,585,952 and United States Patent Publication No. 2010-0173978 concern scFv molecules that are immunospecific for ErbB2. Bi-specific T-cell engagers (“BiTEs”), a type of scFv molecule has been described (WO 05/061547; Baeuerle, P et al. (2008) “BiTE: A New Class Of Antibodies That Recruit T-Cells,” Drugs of the Future 33: 137-147; Bargou, et al. 2008) “Tumor Regression in Cancer Patients by Very Low Doses of a T-Cell-Engaging Antibody,” Science 321:974-977). Such molecules are composed of a single polypeptide chain molecule having two antigen-binding domains, one of which immunospecifically binds to a CD3 epitope and the second of which immunospecifically binds to an antigen present on the surface of a target cell.

The provision of non-mono-specific diabodies provides a significant advantage: the capacity to co-ligate and co-localize different epitopes. Bi-valent diabodies thus have wide-ranging applications including therapy and immunodiagnosis. Bi-valency allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221).

The bi-valency of diabodies has led to their use to co-ligate differing cells, for example, cross-linking cytotoxic T-cells to tumor cells (Staerz et al. (1985) “Hybrid Antibodies Can Target Sites For Attack By T-Cells,” Nature 314:628-631, and Holliger et al. (1996) “Specific Killing Of Lymphoma Cells By Cytotoxic T-Cells Mediated By A Bispecific Diabody,” Protein Eng. 9:299-305). Thus, for example, diabody Epitope-Binding Domains may be directed to a surface determinant of any immune effector cell such as CD3, CD16, CD32, or CD64, which are expressed on T lymphocytes, Natural Killer (NK) cells or other mononuclear cells. In many studies, diabody binding to effector cell determinants, e.g., Fcγ receptors (FcyR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bispecific Antibody Conjugates In Therapeutics,” Adv. Drug. Deliv. Rev. 55:171-197).

However, the above advantages come at salient cost. The formation of such non-mono-specific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-mono-specific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (Takemura, S. et al. (2000) “Construction Of A Diabody (Small Recombinant Bispecific Antibody) Using A Refolding System,” Protein Eng. 13(8):583-588).

The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672). However, the art has recognized that bi-specific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-mono-specific diabodies, termed DART®s (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; and Moore, P. A. et al. (2011) “Application Of Dual Affinity Retargeting Molecules To Achieve Optimal Redirected T-Cell Killing Of B-Cell Lymphoma,” Blood 117 (17): 4542-4551; Veri, M. C. et al. (2010) “Therapeutic Control Of B Cell Activation Via Recruitment Of Fcgamma Receptor IIb(CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species. For example, the addition of a cysteine residue to the c-terminus of such constructs has been shown to allow disulfide bonding between the polypeptide chains, stabilizing the resulting heterodimer without interfering with the binding characteristics of the bi-valent molecule.

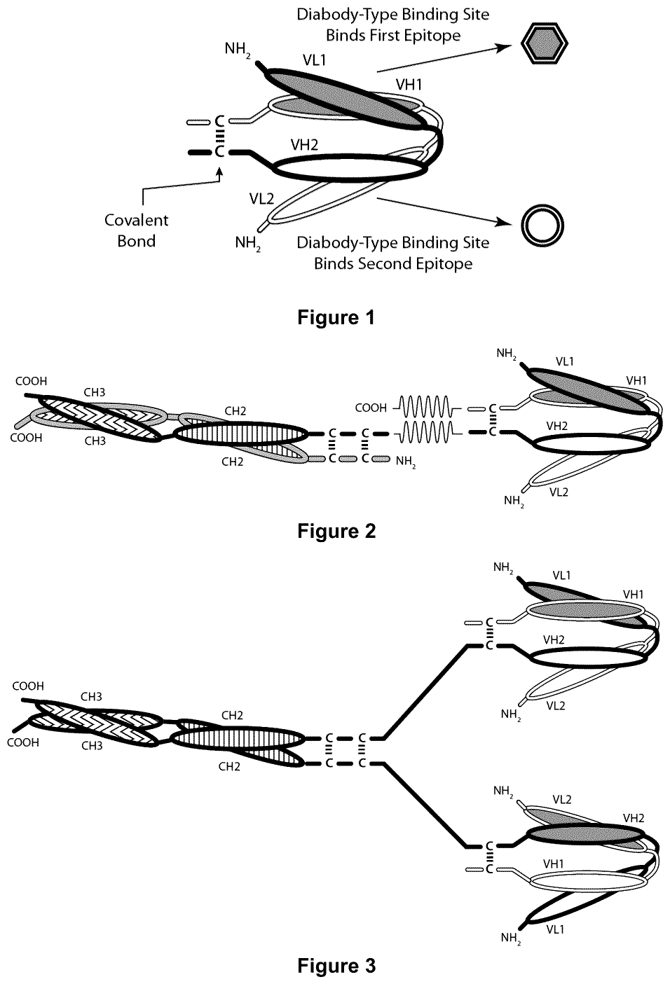

Each of the two polypeptides of the simplest DART® comprises three Domains (FIG. 1). The first polypeptide comprises: (i) a Domain that comprises a binding region of a Light Chain Variable Domain of the a first immunoglobulin (VL1), (ii) a second Domain that comprises a binding region of a Heavy Chain Variable Domain of a second immunoglobulin (VH2), and (iii) a third Domain that serves to promote heterodimerization with the second polypeptide and to covalently bond the first polypeptide to the second polypeptide of the diabody. The second polypeptide contains a complementary first Domain (a VL2 Domain), a complementary second Domain (a VH1 Domain) and a third Domain that complexes with the third Domain of the first polypeptide chain in order to promote heterodimerization and covalent bonding with the first polypeptide chain. Such molecules are stable, potent and have the ability to simultaneously bind two or more antigens.

In one embodiment, the third Domains of the first and second polypeptides each contain a cysteine residue, which serves to bind the polypeptides together via a disulfide bond. The third Domain of one or both of the polypeptides may additionally possesses the sequence of a CH2-CH3 Domain, such that complexing of the diabody polypeptides forms an Fc Domain that is capable of binding to the Fc receptor of cells (such as B lymphocytes, dendritic cells, Natural Killer cells, macrophages, neutrophils, cosinophils, basophils and mast cells) (FIG. 2). Many variations of such molecules have been described (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538). These Fc-bearing DART®s may comprise three polypeptide chains. The first polypeptide of such a diabody contains three Domains: (i) a VL1-containing Domain, (ii) a VH2-containing Domain and (iii) a Domain containing a CH2-CH3 sequence. The second polypeptide of such DART® contains: (i) a VL2-containing Domain, (ii) a VH1-containing Domain and (iii) a Domain that promotes heterodimerization and covalent bonding with the diabody's first polypeptide chain. The third polypeptide of such DART® comprises a CH2-CH3 sequence. Thus, the first and second polypeptide chains of such DART® complex together to form a VL1/VH1 binding site that is capable of binding to the epitope, as well as a VL2/VH2 binding site that is capable of binding to the second epitope. The first and second polypeptides are bonded to one another through a disulfide bond involving cysteine residues in their respective third Domains. Notably, the first and third polypeptide chains complex with one another to form an Fc Domain that is stabilized via a disulfide bond. Such diabodies have enhanced potency. Such Fc-bearing DART®s may have either of two orientations (Table 1):

TABLE 1

| | | |
|---|---|---|
| First | 3^{rd} Chain | NH₂—CH2—CH3—COOH |
| Orientation | 1^{st} Chain | NH₂—VL1—VH2-Heterodimer-Promoting |
| | | Domain-CH2—CH3—COOH |
| | 2^{nd} Chain | NH₂—VL2—VH1-Heterodimer-Promoting Domain-COOH |
| Second | 3^{rd} Chain | NH₂—CH2—CH3—COOH |
| Orientation | 1^{st} Chain | NH₂—CH2—CH3—VL1—VH2-Heterodimer- |
| | | Promoting Domain-COOH |
| | 2^{nd} Chain | NH₂—VL2—VH1-Heterodimer-Promoting Domain-COOH |

An even more complex DART®, termed an Fc-DART® (FIG. 3) has also been described (WO 2012/018687). Fc-DART®s have four polypeptide chains. The first and third polypeptide chains of such a diabody contain three Domains: (i) a VL1-containing Domain, (ii) a VH2-containing Domain and (iii) a Domain containing a CH2-CH3 sequence. The second and fourth polypeptide of the Fc-DART® contain: (i) a VL2-containing Domain, (ii) a VH1-containing Domain and (iii) a Domain that promotes heterodimerization and covalent bonding with the Fc-DART'sTM first polypeptide chain. The VL and/or VH Domains of the third and fourth polypeptide chains, and VL and/or VH Domains of the first and second polypeptide chains may be the same or different so as to permit tetravalent binding that is either mono-specific, bi-specific or tetra-specific (Table 2).

TABLE 2

| | | |
|---|---|---|
| Bi-Specific | 2^{nd} Chain | NH₂—VL2—VH1—COOH |
| | l^{st} Chain | NH₂—VL1—VH2—[CH2—CH3]—COOH |
| | 3^{rd} Chain | NH₂—VL1—VH2—[CH2—CH3]—COOH |
| | 4^{th} Chain | NH₂—VL2—VH1—COOH |
| Tetra-Specific | 2^{nd} Chain | NH₂—VL2—VH1—COOH |
| | 1^{st} Chain | NH₂—VL1—VH2—[CH2—CH3]—COOH |
| | 3^{rd} Chain | NH₂—VL3—VH4—[CH2—CH3]—COOH |
| | 4^{th} Chain | NH₂—VL4—VH3—COOH |

Throughout this application, the numbering of amino acid residues of the light and heavy chains of antibodies is according to the EU index as in Kabat et al. (1992) SEQUENCES OF PROTEINS OF IMMUNOLOGICAL INTEREST, National Institutes of Health Publication No. 91-3242.

The terms “polypeptide” and “peptide” are used interchangeably herein to refer to polymers of amino acids of any length, but especially lengths greater than 3, 5, 10, 15, 20 or 25 amino acid residues. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The polypeptides of this invention can occur as single chains or as complexed chains.

The terms “diabody” and “DART™” have been discussed above. A DART® is a type of diabody that comprises at least two polypeptide chains that preferably complex with one another through a covalent interaction to form at least two epitope-binding sites, which may recognize the same or different epitopes. Two of the polypeptide chains of a diabody or DART™ each comprise immunoglobulin Light Chain Variable Region and an immunoglobulin Heavy Chain Variable Region, but these regions do not interact to form an Epitope-Binding Site (i.e., they are not mutually “complementary”). Rather, the immunoglobulin Heavy Chain Variable Region of one (e.g., the first) of the diabody or DART™ polypeptide chains interacts with the immunoglobulin Light Chain Variable Region of a different (e.g., the second) diabody or DART™ polypeptide chain to form an Epitope-Binding Site. Similarly, the immunoglobulin Light Chain Variable Region of one (e.g., the first) of the diabody or DART™ polypeptide chains interacts with the immunoglobulin Heavy Chain Variable Region of a different (e.g., the second) diabody or DART™ polypeptide chain to form an Epitope-Binding Site. DART™ molecules are disclosed in United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; WO 2006/113665, WO 2008/157379 and Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M.C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62 (7): 1933-1943; and Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399 (3): 436-449.

As used herein, the terms “association” or “associating,” with regard to polypeptides (e.g., one diabody polypeptide to another, an immunoglobulin light chain to an immunoglobulin heavy chain, one CH2-CH3 Domain to another CH2-CH3 Domain, etc.) is intended to denote a non-covalent combining of the polypeptides. The terms “complexes” or “complexing” are intended to denote a covalent combining of the polypeptides.

As used herein, the diabodies of the present invention are said to mediate “coordinated binding” if their Epitope-Binding Domains are capable of concurrently being bound to their respective recognized epitopes. Such binding may be simultaneous.

The Epitope-Binding Domains of the diabodies of the present invention bind to their recognized epitopes in an “immunospecific” manner. As used herein, an antibody, diabody or other epitopc-binding molecule is said to “immunospecifically” bind a region of another molecule (i.e., an epitopc) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moicty or epitopc) that immunospecifically binds to a first target may or may not specifically or preferentially bind to a second target. As such, “specific binding” does not necessarily require (although it can include) exclusive binding. Generally, but not necessarily, reference to binding means “specific” binding.

The various immunoglobulin Domains of such molecules may be derived from immunoglobulins of any isotype or allotype including, but not limited to, IgA, IgD, IgG, IgE and IgM. In preferred embodiments, as discussed below such immunoglobulins are derived from IgG immunoglobulins. In specific embodiments, the IgG isotype used is IgG1, however IgG of other isotypes (e.g., IgG2, IgG3 or IgG4 or an allotype thereof) may be employed.

### II. Preferred PD-1×LAG-3 Bi-Specific Diabodies of the Present Invention

The present invention relates to PD-1×LAG-3 bi-specific diabodies. The preferred PD-1×LAG-3 bi-specific diabodies of the present invention possess epitope-binding fragments of antibodies that enable them to be able to coordinately bind to two different epitopes: an epitope of PD-1 and an epitope of LAG-3, so as to attenuate the inhibitory activities of such molecules. As used herein, such attenuation refers to a decrease of at least 20%, a decrease of at least 50%, a decrease of at least 80%, or a decrease of at least 90% in detectable PD-1 and or LAG-3 inhibitory activity, or the complete elimination of detectable PD-1 and or LAG-3 inhibitory activity.

### A. Anti-PD-1 Binding Capabilities

Antibodies that are immunospecific for PD-1 are known (see, e.g., U.S. Pat. No. 8,008,449; 8,552,154; PCT Patent Publications WO 2012/135408; WO 2012/145549; and WO 2013/014668). Additional desired antibodies may be made by isolating antibody-secreting hybridomas elicited using PD-1 or a peptide fragment thereof. Human PD-1 (including a 20 amino acid residue signal sequence (shown underlined) and the 268 amino acid residue mature protein) has the amino acid sequence (SEQ ID NO:1):

MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

Preferred anti-PD-1 antibodies include: PD-1 mAb 1 (5C4; BMS-936558)), PD-1 mAb 2 (MK-3475; Merck, lambrolizumab), PD-1 mAb 3 (EH12.2H7; Dana Farber) and PD-1 mAb 4 (CT-011; CureTech, BAT-1).

The amino acid sequence of the Heavy Chain Variable Domain of PD-1 mAb 1 has the amino acid sequence (SEQ ID NO:2) (CDRs are shown underlined):

QVQLVESGGG VVQPGRSLRL DCKASGITFS NSGMHWVRQA
PGKGLEWVAV IWYDGSKRYY ADSVKGRFTI SRDNSKNTLF
LQMNSLRAED TAVYYCATND DYWGQGTLVT VSS

The amino acid sequence of the Light Chain Variable Domain of PD-1 mAb 1 has the amino acid sequence (SEQ ID NO:3) (CDRs are shown underlined):

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIK

The amino acid sequence of the Heavy Chain Variable Domain of PD-1 mAb 2 has the amino acid sequence (SEQ ID NO:4) (CDRs are shown underlined):

QVQLVQSGVE VKKPGASVKV SCKASGYTFT NYYMYWVRQA
PGQGLEWMGG INPSNGGTNF NEKFKNRVTL TTDSSITTAY
MELKSLQFDD TAVYYCARRD YRFDMGFDYW GQGTTVTVSS

The amino acid sequence of the Light Chain Variable Domain of PD-1 mAb 2 has the amino acid sequence (SEQ ID NO:5) (CDRs are shown underlined):

EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEI K

The amino acid sequence of the Heavy Chain Variable Domain of PD-1 mAb 3 has the amino acid sequence (SEQ ID NO:6) (CDRs are shown underlined):

QVQLQQSGAE LAKPGASVQM SCKASGYSFT SSWIHWVKQR
PGQGLEWIGY IYPSTGFTEY NQKFKDKATL TADKSSSTAY
MQLSSLTSED SAVYYCARWR DSSGYHAMDY WGQGTSVTVSS

The amino acid sequence of the Light Chain Variable Domain of PD-1 mAb 3 has the amino acid sequence (SEQ ID NO:7) (CDRs are shown underlined):

DIVLTQSPAS LTVSLGQRAT ISCRASQSVS TSGYSYMHWY
QQKPGQPPKL LIKFGSNLES GIPARFSGSG SGTDFTLNIH
PVEEEDTATY YCQHSWEIPY TFGGGTKLEI K

The amino acid sequence of the Heavy Chain Variable Domain of PD-1 mAb 4 has the amino acid sequence (SEQ ID NO:8) (CDRs are shown underlined):

QVQLVQSGSE LKKPGASVKI SCKASGYTFT NYGMNWVRQA
PGQGLQWMGW INTDSGESTY AEEFKGRFVF SLDTSVNTAY
LQITSLTAED TGMYFCVRVG YDALDYWGQG TLVTVSS

The amino acid sequence of the Light Chain Variable Domain of PD-1 mAb 4 has the amino acid sequence (SEQ ID NO:9) (CDRs are shown underlined):

EIVLTQSPSS LSASVGDRVT ITCSARSSVS YMHWFQQKPG
KAPKLWIYRT SNLASGVPSR FSGSGSGTSY CLTINSLQPE
DFATYYCQQR SSFPLTFGGG TKLEIK

### B. Anti-LAG-3 Binding Capabilities

Antibodies that are immunospecific for LAG-3 are also known (see, e.g., WO 2014/008218). Additional desired antibodies may be made by isolating antibody-secreting hybridomas elicited using LAG-3 or a peptide fragment thereof. Human LAG-3 (including a 28 amino acid residue signal sequence (shown underlined) and the 497 amino acid residue mature protein) has the amino acid sequence (SEQ ID NO:10):

MWEAQFLGLL FLQPLWVAPV KPLQPGAEVP VVWAQEGAPA
QLPCSPTIPL QDLSLLRRAG VTWQHQPDSG PPAAAPGHPL
APGPHPAAPS SWGPRPRRYT VLSVGPGGLR SGRLPLQPRV
QLDERGRQRG DFSLWLRPAR RADAGEYRAA VHLRDRALSC
RLRLRLGQAS MTASPPGSLR ASDWVILNCS FSRPDRPASV
HWFRNRGQGR VPVRESPHHH LAESFLFLPQ VSPMDSGPWG
CILTYRDGFN VSIMYNLTVL GLEPPTPLTV YAGAGSRVGL
PCRLPAGVGT RSFLTAKWTP PGGGPDLLVT GDNGDFTLRL
EDVSQAQAGT YTCHIHLQEQ QLNATVTLAI ITVTPKSFGS
PGSLGKLLCE VTPVSGQERF VWSSLDTPSQ RSFSGPWLEA
QEAQLLSQPW QCQLYQGERL LGAAVYFTEL SSPGAQRSGR
APGALPAGHL LLFLILGVLS LLLLVTGAFG FHLWRRQWRP
RRFSALEQGI HPPQAQSKIE ELEQEPEPEP EPEPEPEPEP EPEQL

A preferred anti-LAG-3 antibody is LAG-3 mAb 1 (25F7; BMS-986016, Medarex/BMS). The amino acid sequence of the Heavy Chain Variable Domain of LAG-3 mAb 1 has the amino acid sequence (SEQ ID NO:11) (CDRs are shown underlined):

QVQLQQWGAG LLKPSETLSL TCAVYGGSFS DYYWNWIRQP
PGKGLEWIGE INHNGNTNSN PSLKSRVTLS LDTSKNQFSL
KLRSVTAADT AVYYCAFGYS DYEYNWFDPW GQGTLVTVSS

The amino acid sequence of the Light Chain Variable Domain of LAG-3 mAb 1 has the amino acid sequence (SEQ ID NO:12) (CDRs are shown underlined):

EIVLTQSPAT LSLSPGERAT LSCRASQSIS SYLANYQQKP
GQAPRLLIYD ASNRATGIPA RFSGSGSGTD FTLTISSLEP
EDFAVYYCQQ RSNWPLTFGQ GTNLEIK

As used herein, the term “epitope-binding fragment of an antibody” means a fragment of an antibody capable of immunospecifically binding to an epitope. An epitope-binding fragment may contain 1, 2, 3, 4, 5 or all 6 of the CDR Domains of such antibody and, although capable of immunospecifically binding to such epitope, may exhibit an immunospecificity, affinity or selectivity toward such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxyl terminus (e.g., a diabody, an Fab fragment, an Fab₂ fragment, etc.).

### C. Preferred DART® Diabodies of the Present Invention

### 1. General Considerations

The preferred diabodies of the present invention are bi-specific, tetra-valent, Fc-DART® diabodies (FIG. 3) that are composed of four total polypeptide chains. The four polypeptide chains comprise two CH2-CH3-containing polypeptides (i.e., the “first” and “third” polypeptide chains of the diabody) that complex together to form an Fc Domain and two identical non-CH3-containing polypeptides (i.e., the “second” and “fourth” polypeptide chains of the diabody), each of which complex with a CH2-CH3-containing polypeptide of the diabody to form an Epitope-Binding Domain that is immunospecific for PD-1 or LAG-3. Thus, for example, the first polypeptide chain will contain an anti-PD-1 (or anti-LAG-3) Variable Light Chain (VL) Domain and an anti-LAG-3 (or anti-PD-1) Variable Heavy Chain (VH) Domain, and will complex with a second polypeptide chain that possesses a complementary anti-PD-1 (or anti-LAG-3) VH Domain and a complementary anti-LAG-3 (or anti-PD-1) VL Domain, so as to form a first pair of PD-1 and LAG-3 Epitope-Binding Domains. Likewise, the third polypeptide chain will contain an anti-PD-1 (or an anti-LAG-3) Variable Light Chain (VL) Domain and an anti-LAG-3 (or an anti-PD-1) Variable Heavy Chain (VH) Domain, and will complex with a fourth polypeptide chain that possesses a complementary anti-PD-1 (or anti-LAG-3) VH Domain and a complementary anti-LAG-3 (or anti-PD-1) VL Domain, so as to form a second pair of PD-1 and LAG-3 Epitope-Binding Domains.

Since the Variable Light Chain and Variable Heavy Chain Domains of the same polypeptide are directed toward different epitopes, they cannot complex together to form an Epitope-Binding Domain that is able to bind either PD-1 or LAG-3. The Variable Light Chain and Variable Heavy Chain Domains of the first polypeptide are preferably spaced apart from one another by an intervening linker peptide that is sufficiently short as to substantially prevent the complexing of these Domains. An exemplary linker, has the sequence (SEQ ID NO:13: GGGSGGGG. Other linkers of similar length (so as to prevent the VL and VH Domains of the same polypeptide chain from interacting with each other) may alternatively be employed.

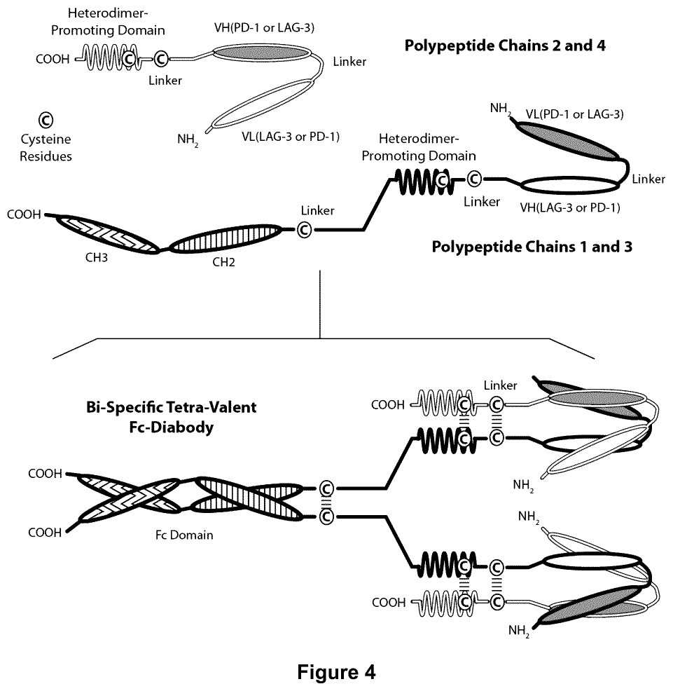

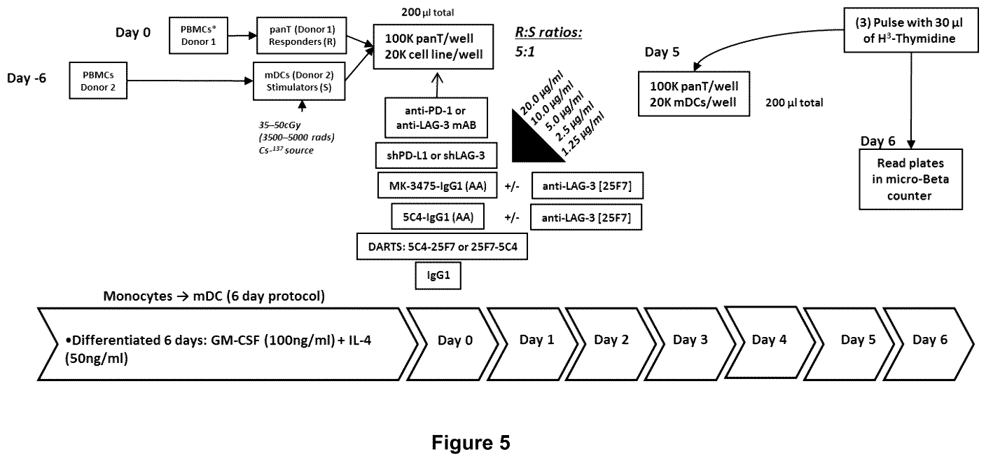

As shown in FIG. 4, the most preferred bi-specific, tetra-valent, Fc-DART® diabodies of the present invention are Fc-DART® diabodies that have been modified to contain Heterodimer-Promoting Domains. The inclusion of such Domains fosters heterodimer formation between the polypeptide chains of the diabody. The inclusion of such Domains is not essential, and the present invention includes PD-1×LAG-3 bi-specific diabodies that do not possess such Domains. Preferred Heterodimer-Promoting Domains include “E-coil” Domains (SEQ ID NO:14): EVAALEKEVAALEKEVAALEKEVAALEK, and “K-coil” Domains (SEQ ID NO:15): KVAALKEKVAALKEKVAALKEKVAALKE. More specifically, a pair of polypeptide chains that are desired to complex together are each engineered to contain one (or more) such Heterodimer-Promoting Domains, with the employed Domain(s) of one polypeptide chain being complementary to the employed Domain(s) of the other polypeptide chain (e.g., one polypeptide chain will contain an E-coil Domain and the other will contain a K-coil Domain). Where the two polypeptide chains are engineered to contain more than one such Heterodimer-Promoting Domains, they can be of the same charge, or more preferably of opposite charge.

Particularly preferred are Heterodimer-Promoting Domains that comprise modifications of the above-described E-coil and K-coil sequences so as to include one or more cysteine residues. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred arc Heterodimer-Promoting Domains include a Modified E-Coil having the amino acid sequence (SEQ ID NO:16): EVAACEKEVAALEKEVAALEKEVAALEK, wherein a leucine residue of SEQ ID NO:14 has been replaced with a cysteine residue (shown underlined), and a Modified K-Coil the amino having acid sequence (SEQ ID NO:17): KVAACKEKVAALKEKVAALKEKVAALKE, wherein a leucine residue of SEQ ID NO:15 has been replaced with a cysteine residue (shown underlined).

The Variable Heavy Chain Domain of the first polypeptide and the Heterodimer-Promoting Domain of that polypeptide are preferably spaced apart from one another by an intervening linker peptide that contains 1, 2, 3 or more cysteine residues. A preferred cysteine-containing spacer peptide has the sequence is SEQ ID NO:18: GGCGGG. Other linkers of similar length (so as to include a cysteine residue that can covalently bond with a cysteine residue of another polypeptide chain) may be employed.

Preferably, the employed Heterodimer-Promoting Domain and the CH2-CH3Domain of the first polypeptide chain are spaced apart from one another by an intervening cysteine-containing linker peptide that provides improved stabilization to the Heterodimer-Promoting Domain. A suitable cysteine-containing linker peptide has the amino acid sequence (SEQ ID NO:19): DKTHTCPPCP, however, a more preferred linker has the amino acid sequence (SEQ ID NO:20): LEPKSADKTHTCPPC. Other linkers of similar length (so as to include a cysteine residue that can covalently bond with a cysteine residue of another polypeptide chain) may alternatively be employed.

The amino acid sequence of a wild-type CH2-CH3 Domain is as follows (positioning is as in the EU index as in Kabat et al. (1992) SEQUENCES OF PROTEINS OF IMMUNOLOGICAL INTEREST, National Institutes of Health Publication No. 91-3242) (SEQ ID NO:21):

```
|CH2→
PAPELLGGPS VFLFPPKPKD TLMISRTPEV TCVVVDVSHE
230 240 250 260
DPEVKFNWYV DGVEVHNAKT KPREEQYNST YRVVSVLTVL
270 280 290 300
                                                            ←CH2|CH3→
HQDWLNGKEY KCKVSNKALP APIEKTISKA K GQPREPQVY
310 320 330 340
TLPPSREEMT KNQVSLTCLV KGFYPSDIAV EWESNGQPEN
350 360 370 380
NYKTTPPVLD SDGSFFLYSK LTVDKSRWQQ GNVFSCSVMH
390 400 410 420
                            ←CH3|
EALHNHYTQK SLSLSPGK
430 440
```

For tri-specific or tetra-specific diabodies (i.e., for diabodies whose first and third polypeptide chains are not identical), it is desirable to reduce or prevent homodimerization from occurring between the CH2-CH3 Domains of two first polypeptide chains or between the CH2-CH3 Domains of two third polypeptide chains. In order to promote heterodimerization between the first and third polypeptide chains, the CH2-CH3 Domain of these chains are preferably modified so as to promote such heterodimerization. For example, an amino acid substitution (preferably a substitution with an amino acid comprising a bulky side group forming a ‘knob’, e.g., tryptophan) can be introduced into the CH2 or CH3 Domain of the first polypeptide chain such that steric interference will prevent interaction with a similarly mutated Domain and will obligate the mutated Domain to pair with a Domain into which a complementary, or accommodating mutation has been engineered, i.e., ‘the hole’ (e.g., a substitution with glycine). Such sets of mutations can be engineered into any pair of polypeptides comprising the diabody molecule, and further, engineered into any portion of the polypeptides chains of said pair. Methods of protein engineering to favor heterodimerization over homodimerization are well-known in the art, in particular with respect to the engineering of immunoglobulin-like molecules, and are encompassed herein (see e.g., U.S. Pat. No. 7,695,936 and Patent Publication 2007/0196363, Ridgway et al. (1996) “'Knobs-Into-Holes' Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270:26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety. A preferred knob is created by modifying a native IgG Fc Domain to contain the modification T366W. A preferred hole is created by modifying a native IgG Fc Domain to contain the modification T366S, L368A and Y407V. To aid in purifying the diabodies of the present invention, the polypeptide chain containing the hole mutations additionally comprises a substitution at position 435(H435R) to remove the Protein A binding site. Thus, homodimers of polypeptides containing the hole mutations will not bind to protein A, whereas the diabodies that form as a result of knob and hole containing heterodimers will retain its ability to bind protein A via the protein A binding site on the polypeptide chain containing the knob mutation.

The invention also encompasses molecules comprising variant Fc Domains comprising one or more amino acid substitutions, insertions, or deletions relative to a comparable wild-type Fc Domain. Molecules comprising variant Fc Domains normally have altered phenotypes relative to molecules comprising wild-type Fc Domains The variant phenotype may be expressed as altered serum half-life, altered stability, altered susceptibility to cellular enzymes or altered effector function as assayed in an NK dependent or macrophage dependent assay. Fc Domain modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57 (18): 8882-8890). Exemplary variants of human IgG1 Fc Domains with reduced binding to CD32B and/or increased binding to CD16A contain F243L, R929P, Y300L, V3051 or P296L substitutions. These amino acid substitutions may be present in a human IgG1 Fc Domain in any combination. In one embodiment, the human IgG1 Fc Domain variant contains a F243L, R929P and Y300L substitution. In another embodiment, the human IgG1 Fc Domain variant contains a F243L, R929P, Y300L, V305I and P296L substitution. In another embodiment, the human IgG1 Fc Domain variant contains an N297Q substitution, L234A and L235A substitutions or a D265A substitution, as these mutations abolish FcR binding. The CH2-CH3 Domain of the first polypeptide chain of such molecules will have the “knob-bearing” sequence (SEQ ID NO:22):

PAPEAAGGPS VFLFPPKPKD TLMISRTPEV TCVVVDVSHE
DPEVKFNWYV DGVEVHNAKT KPREEQYNST YRVVSVLTVL
HQDWLNGKEY KCKVSNKALP APIEKTISKA KGQPREPQVY
TLPPSREEMT KNQVSLWCLV KGFYPSDIAV EWESNGQPEN
NYKTTPPVLD SDGSFFLYSK LTVDKSRWQQ GNVFSCSVMH
EALHNHYTQK SLSLSPGK

or the “hole-bearing” sequence with an H435R substitution to abrogate Protein A binding (SEQ ID NO:23):

PAPEAAGGPS VFLFPPKPKD TLMISRTPEV TCVVVDVSHE
DPEVKFNWYV DGVEVHNAKT KPREEQYNST YRVVSVLTVL
HQDWLNGKEY KCKVSNKALP APIEKTISKA KGQPREPQVY
TLPPSREEMT KNQVSLSCAV KGFYPSDIAV EWESNGQPEN
NYKTTPPVLD SDGSFFLVSK LTVDKSRWQQ GNVFSCSVMH
EALHNRYTQK SLSLSPGK

As will be recognized, a “hole-bearing” CH2-CH3 Domain (e.g., SEQ ID NO:22) could be employed in the first polypeptide chain, in which case, a “knob-bearing” CH2-CH3 Domain (e.g., SEQ ID NO:22) would be employed in the third polypeptide chain.

For bi-specific, tetra-valent, diabodies of the present invention whose first and third polypeptide chains are not different, a preferred CH2-CH3 Domain is a Modified CH2-CH3Domain having the amino acid sequence (SEQ ID NO:24):

PAPEAAGGPS VFLFPPKPKD TLYITREPEV TCVVVDVSHE
DPEVKFNWYV DGVEVHNAKT KPREEQYNST YRVVSVLTVL
HQDWLNGKEY KCKVSNKALP APIEKTISKA KGQPREPQVY
TLPPSREEMT KNQVSLTCLV KGFYPSDIAV EWESNGQPEN
NYKTTPPVLD SDGSFFLYSK LTVDKSRWQQ GNVFSCSVMH
EALHNHYTQK SLSLSPG

Thus, in sum, the preferred first and third polypeptide chains of a preferred PD-1×LAG-3 bi-specific, tetra-valent, diabody Fc-DART@ diabody of the present invention have identical sequences, and the second and fourth polypeptide chains of such preferred PD-1×LAG-3 bi-specific, tetra-valent, Fc-DART@ diabodies have identical sequences, as shown in Table 3:

TABLE 3

| | Domains of the First and Third | Domains of the Second and Fourth |
|---|---|---|
| Variation | Polypeptide Chains | Polypeptide Chains |
| I | (VL of anti-PD-1 Epitope-Binding | (VL of anti-LAG-3 Epitope Binding |
| | Domain) - (Linker) - (VH of anti- | Domain) - (Linker) - (VH of an anti-PD-1 |
| | LAG-3 Epitope-Binding Domain) - | Epitope Binding Domain) - (Linker) - |
| | (Linker) - (Modified E-Coil | (Modified K-Coil Heterodimer-Promoting |
| | Heterodimer-Promoting Domain) - | Domain) |
| | (Linker) - (Modified CH2—CH3 | |
| | Domain) | |
| II | (VL of anti-PD-1 Epitope Binding | (VL of anti-LAG-3 Epitope Binding |
| | Domain) - (Linker) - (VH of anti- | Domain) - (Linker) - (VH of an anti-PD-1 |
| | LAG-3 Epitope Binding Domain) - | Epitope Binding Domain) - (Linker) - |
| | (Linker) - (Modified K-Coil | (Modified E-Coil Heterodimer-Promoting |
| | Heterodimer-Promoting Domain) - | Domain) |
| | (Linker) - (Modified CH2—CH3 | |
| | Domain) | |
| III | (VL of anti-LAG-3 Epitope Binding | (VL of anti-PD-1 Epitope Binding Domain) - |
| | Domain) - (Linker) - (VH of anti-PD- | (Linker) - (VH of an anti-LAG-3 |
| | 1 Epitope Binding Domain) - | Epitope Binding Domain) - (Linker) - |
| | (Linker) - (Modified E-Coil | (Modified K-Coil Heterodimer-Promoting |
| | Heterodimer-Promoting Domain) - | Domain) |
| | (Linker) - (Modified CH2—CH3 | |
| | Domain) | |
| IV | (VL of anti-LAG-3 Epitope Binding | (VL of anti-PD-1 Epitope Binding Domain) - |
| | Domain) - (Linker) - (VH of anti-PD- | (Linker) - (VH of an anti-LAG-3 |
| | 1 Epitope Binding Domain) - | Epitope Binding Domain) - (Linker) - |
| | (Linker) - (Modified K-Coil | (Modified E-Coil Heterodimer-Promoting |
| | Heterodimer-Promoting Domain) - | Domain) |
| | (Linker) - (Modified CH2—CH3 | |
| | Domain) | |

### 2. First Exemplary PD-1×LAG-3 Bi-Specific Fc-DART® Diabody (PD-1×LAG-3 Fc-DART®-1)

A first particularly preferred PD-1×LAG-3 bi-specific, tetra-valent, Fc-DART® diabody of the present invention (“ PD-1×LAG-3 Fc-DART®-1”) has first and third polypeptide chains of identical sequence. Such first/third polypeptide chain has the amino acid sequence (SEQ ID NO:25):

EIVLTQSPAT LSLSPGERAT LSCRASQSIS SYLAWYQQKP
GQAPRLLIYD ASNRATGIPA RFSGSGSGTD FTLTISSLEP
EDFAVYYCQQ RSNWPLTFGQ GTNLEIKGGG SGGGGQVQLV
ESGGGVVQPG RSLRLDCKAS GITFSNSGMH WVRQAPGKGL
EWVAVIWYDG SKRYYADSVK GRFTISRDNS KNTLFLQMNS
LRAEDTAVYY CATNDDYWGQ GTLVTVSSGG CGGGEVAACE
KEVAALEKEV AALEKEVAAL EKLEPKSADK THTCPPCPAP
EAAGGPSVFL FPPKPKDTLY ITREPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLTCLVKGF YPSDIAVEWE SNGQPENNYK
TTPPVLDSDG SFFLYSKLTV DKSRWQQGNV FSCSVMHEAL
HNHYTQKSLS LSPG

wherein, amino acid residues 1-107 are the amino acid residues of the Light Chain Variable Domain of LAG-3 mAb 1 (SEQ ID NO:12), amino acid residues 108-115 are the amino acid residues of the linker GGGSGGGG (SEQ ID NO:13), amino acid residues 116-228 are the amino acid residues of the Heavy Chain Variable Domain of PD-1 mAb 1 (SEQ ID NO:2), amino acid residues 229-234 are the amino acid residues of the cysteine-containing spacer peptide GGCGGG (SEQ ID NO:18), amino acid residues 235-262 are the amino acid residues of the Modified E-Coil (SEQ ID NO:16), amino acid residues 263-277 are the amino acid residues of the cysteine-containing linker peptide LEPKSADKTHTCPPC (SEQ ID NO:20) and amino acid residues 278-494 are the amino acid residues of the Modified CH2-CH3 Domain (SEQ ID NO:24).

A nucleic acid molecule that encodes such a first/third polypeptide chain is (SEQ ID NO: 26):

gaaattgtcc tgacacagtc tcccgcaacc ctgagtttga
gtcctgggga gcgagcaact ctctcctgcc gagcctccca
gagtatctcc tcctacctcg cctggtacca acagaagcca
gggcaggctc caaggctgct tatctatgac gcctctaacc
gcgcaactgg gattcccgca cgcttctccg gctctggttc
cggcacagac tttacactta ctatctctag cctggagcca
gaagactttg ccgtgtacta ttgtcagcaa cgttccaatt
ggccccttac ctttgggcag ggcactaact tggaaatcaa
aggtggcgga tccggcggcg gaggccaggt tcagctggtc
gagagtggtg gcggcgttgt gcaacctggg cgttccctcc
gattggactg taaagcttcc ggcattactt tctcaaattc
cggcatgcat

gaaattgtcc tgacacagtc tcccgcaacc ctgagtttga
gtcctgggga gcgagcaact ctctcctgcc gagcctccca
gagtatctcc tcctacctcg cctggtacca acagaagcca
gggcaggctc caaggctgct tatctatgac gcctctaacc
gcgcaactgg gattcccgca cgcttctccg gctctggttc
cggcacagac tttacactta ctatctctag cctggagcca
gaagactttg ccgtgtacta ttgtcagcaa cgttccaatt
ggccccttac ctttgggcag ggcactaact tggaaatcaa
aggtggcgga tccggcggcg gaggccaggt tcagctggtc
gagagtggtg gcggcgttgt gcaacctggg cgttccctcc
gattggactg taaagcttcc ggcattactt tctcaaattc
cggcatgcat tgggtgaggc aagcccctgg aaaagggctc
gaatgggtgg ctgtgatttg gtacgatggc agcaaacggt
actacgccga ttctgttaag ggccgcttta ccatctcccg
cgataactca aagaacacac tgtttctgca aatgaatagt
cttagagccg aggacaccgc cgtgtactac tgtgccacaa
atgacgatta ttgggggcag ggcacattgg tcacagtgtc
ttccggagga tgtggcggtg gagaagtggc cgcatgtgag
aaagaggttg ctgctttgga gaaggaggtc gctgcacttg
aaaaggaggt cgcagccctg gagaaactgg agcccaaatc
tgctgacaaa actcacacat gcccaccgtg cccagcacct
gaagccgcgg ggggaccgtc agtcttcctc ttccccccaa
aacccaagga caccctctat atcacccggg agcctgaggt
cacatgcgtg gtggtggacg tgagccacga agaccctgag
gtcaagttca actggtacgt ggacggcgtg gaggtgcata
atgccaagac aaagccgcgg gaggagcagt acaacagcac
gtaccgtgtg gtcagcgtcc tcaccgtcct gcaccaggac
tggctgaatg gcaaggagta caagtgcaag gtctccaaca
aagccctccc agcccccatc gagaaaacca tctccaaagc
caaagggcag ccccgagaac cacaggtgta caccctgccc
ccatcccggg aggagatgac caagaaccag gtcagcctga
cctgcctggt caaaggcttc tatcccagcg acatcgccgt
ggagtgggag agcaatgggc agccggagaa caactacaag
accacgcctc ccgtgctgga ctccgacggc tccttcttcc
tctacagcaa gctcaccgtg gacaagagca ggtggcagca
ggggaacgtc ttctcatgct ccgtgatgca tgaggctctg
cacaaccact acacgcagaa gagcctctcc ctgtctccgg gt

The second and fourth polypeptide chain of such PD-1×LAG-3 Fc-DART®-1 have identical sequences. Such second/fourth polypeptide chain has the amino acid sequence (SEQ ID NO: 27):

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIKGGG SGGGGQVQLQ
QWGAGLLKPS ETLSLTCAVY GGSFSDYYWN WIRQPPGKGL
EWIGEINHNG NTNSNPSLKS RVTLSLDTSK NQFSLKLRSV
TAADTAVYYC AFGYSDYEYN WFDPWGQGTL VTVSSGGCGG
GKVAACKEKV AALKEKVAAL KEKVAALKE

wherein, amino acid residues 1-107 are the amino acid residues of the Light Chain Variable Domain of PD-1 mAb 1 (SEQ ID NO:3), amino acid residues 108-115 are the amino acid residues of the linker GGGSGGGG (SEQ ID NO:13), amino acid residues 116-235 are the amino acid residues of the Heavy Chain Variable Domain of LAG-3 mAb 1 (SEQ ID NO:11), amino acid residues 236-241 are the amino acid residues of the cysteine-containing spacer peptide GGCGGG (SEQ ID NO:18), and amino acid residues 242-269 are the amino acid residues of the Modified K-Coil (SEQ ID NO:17).

A nucleic acid molecule that encodes such a second/fourth polypeptide chain is (SEQ ID NO: 28):

gagatcgtac ttacccagtc tcccgccacc ctttccctga
gtcctggtga gcgggccact ctttcctgtc gcgcaagcca
atcagtttct agctacctcg catggtatca gcagaagcca
gggcaggcac ccaggcttct catctatgac gccagtaacc
gcgcaaccgg gatacctgct agattttccg gcagtggatc
tgggaccgat ttcacactga caatttcatc cttggaacca
gaagatttcg cagtctacta ctgccagcaa tcttccaact
ggccaagaac tttcggacag gggaccaaag tggaaattaa
aggtggcgga tccggcggcg gaggccaggt ccagctccag
caatggggag ccgggctgct gaaaccctct gaaacactga
gtctcacatg tgccgtttat ggaggttcct tctccgatta
ttactggaac tggattcgtc agcctcccgg caagggcctg
gagtggatcg gtgagattaa ccacaatggc aataccaata
gcaatcctag tttgaaatct cgcgtcactc tttccctcga
tacaagcaaa aaccagtttt ctttgaaatt gcgatctgta
actgctgctg atactgccgt gtattactgc gcattcggct
actccgacta tgaatataat tggttcgatc cttggggaca
gggaacattg gtaaccgtgt catccggagg atgtggcggt
ggaaaagtgg ccgcatgtaa ggagaaagtt gctgctttga
aagagaaggt cgccgcactt aaggaaaagg tcgcagccct
gaaagag

### 3. Second Exemplary PD-1×LAG-3 Bi-Specific Fc-DART® Diabody (PD-1×LAG-3 Fc-DART®-2)

A second particularly preferred PD-1×LAG-3 bi-specific, tetra-valent, Fc-DART® diabody of the present invention (PD-1×LAG-3 Fc-DART®-2) has first and third polypeptide chains of identical sequence. Such first/third polypeptide chain has the amino acid sequence (SEQ ID NO:29):

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIKGGG SGGGGQVQLQ
QWGAGLLKPS ETLSLTCAVY GGSFSDYYWN WIRQPPGKGL
EWIGEINHNG NTNSNPSLKS RVTLSLDTSK NQFSLKLRSV
TAADTAVYYC AFGYSDYEYN WFDPWGQGTL VTVSSGGCGG
GEVAACEKEV AALEKEVAAL EKEVAALEKL EPKSADKTHT
CPPCPAPEAA GGPSVFLFPP KPKDTLYITR EPEVTCVVVD
VSHEDPEVKF NWYVDGVEVH NAKTKPREEQ YNSTYRVVSV
LTVLHQDWLN GKEYKCKVSN KALPAPIEKT ISKAKGQPRE
PQVYTLPPSR EEMTKNQVSL TCLVKGFYPS DIAVEWESNG
QPENNYKTTP PVLDSDGSFF LYSKLTVDKS RWQQGNVFSC
SVMHEALHNH YTQKSLSLSP G

wherein, amino acid residues 1-107 are the amino acid residues of the Light Chain Variable Domain of PD-1 mAb 1 (SEQ ID NO:3), amino acid residues 108-115 are the amino acid residues of the linker GGGSGGGG (SEQ ID NO:13), amino acid residues 116-235 are the amino acid residues of the Heavy Chain Variable Domain of LAG-3 mAb 1 (SEQ ID NO:11), amino acid residues 236-241 are the amino acid residues of the cysteine-containing spacer peptide GGCGGG (SEQ ID NO:18), amino acid residues 242-269 are the amino acid residues of the Modified E-Coil (SEQ ID NO:16), amino acid residues 270-284 are the amino acid residues of the cysteine-containing linker peptide LEPKSADKTHTCPPC (SEQ ID NO:20) and amino acid residues 285-501 are the amino acid residues of the Modified CH2-CH3 Domain (SEQ ID NO:24).

A nucleic acid molecule that encodes such a first/third polypeptide chain is (SEQ ID NO: 30):

gagatcgtac ttacccagtc tcccgccacc ctttccctga
gtcctggtga gcgggccact ctttcctgtc gcgcaagcca
atcagtttct agctacctcg catggtatca gcagaagcca
gggcaggcac ccaggcttct catctatgac gccagtaacc
gcgcaaccgg gatacctgct agattttccg gcagtggatc
tgggaccgat ttcacactga caatttcatc cttggaacca
gaagatttcg cagtctacta ctgccagcaa tcttccaact
ggccaagaac tttcggacag gggaccaaag tggaaattaa
aggtggcgga tccggcggcg gaggccaggt ccagctccag
caatggggag ccgggctgct gaaaccctct gaaacactga
gtctcacatg tgccgtttat ggaggttcct tctccgatta
ttactggaac tggattcgtc agcctcccgg caagggcctg
gagtggatcg gtgagattaa ccacaatggc aataccaata
gcaatcctag tttgaaatct cgcgtcactc tttccctcga
tacaagcaaa aaccagtttt ctttgaaatt gcgatctgta
actgctgctg atactgccgt gtattactgc gcattcggct
actccgacta tgaatataat tggttcgatc cttggggaca
gggaacattg gtaaccgtgt catccggagg atgtggcggt
ggagaagtgg ccgcatgtga gaaagaggtt gctgctttgg
agaaggaggt cgctgcactt gaaaaggagg tcgcagccct
ggagaaactg gagcccaaat ctgctgacaa aactcacaca
tgcccaccgt gcccagcacc tgaagccgcg gggggaccgt
cagtcttcct cttcccccca aaacccaagg acaccctcta
tatcacccgg gagcctgagg tcacatgcgt ggtggtggac
gtgagccacg aagaccctga ggtcaagttc aactggtacg
tggacggcgt ggaggtgcat aatgccaaga caaagccgcg
ggaggagcag tacaacagca cgtaccgtgt ggtcagcgtc
ctcaccgtcc tgcaccagga ctggctgaat ggcaaggagt
acaagtgcaa ggtctccaac aaagccctcc cagcccccat
cgagaaaacc atctccaaag ccaaagggca gccccgagaa
ccacaggtgt acaccctgcc cccatcccgg gaggagatga
ccaagaacca ggtcagcctg acctgcctgg tcaaaggctt
ctatcccagc gacatcgccg tggagtggga gagcaatggg
cagccggaga acaactacaa gaccacgcct cccgtgctgg
actccgacgg ctccttcttc ctctacagca agctcaccgt
ggacaagagc aggtggcagc aggggaacgt cttctcatgc
tccgtgatgc atgaggctct gcacaaccac tacacgcaga
agagcctctc cctgtctccg ggt

The second and fourth polypeptide chain of PD-1×LAG-3 Fc-DART®-2 have identical sequences. Such second/fourth polypeptide chain has the amino acid sequence (SEQ ID NO: 31):

EIVLTQSPAT LSLSPGERAT LSCRASQSIS SYLAWYQQKP
GQAPRLLIYD ASNRATGIPA RFSGSGSGTD FTLTISSLEP
EDFAVYYCQQ RSNWPLTFGQ GTNLEIKGGG SGGGGQVQLV
ESGGGVVQPG RSLRLDCKAS GITFSNSGMH WVRQAPGKGL
EWVAVIWYDG SKRYYADSVK GRFTISRDNS KNTLFLQMNS
LRAEDTAVYY CATNDDYWGQ GTLVTVSSGG CGGGKVAACK
EKVAALKEKV AALKEKVAAL KE

wherein, amino acid residues 1-107 are the amino acid residues of the Light Chain Variable Domain of LAG-1 mAb 1 (SEQ ID NO:12), amino acid residues 108-115 are the amino acid residues of the linker GGGSGGGG (SEQ ID NO:13), amino acid residues 116-228 are the amino acid residues of the Heavy Chain Variable Domain of PD-1 mAb 1 (SEQ ID NO:2), amino acid residues 229-234 are the amino acid residues of the cysteine-containing spacer peptide GGCGGG (SEQ ID NO:18), and amino acid residues 235-262 are the amino acid residues of the Modified K-Coil (SEQ ID NO:17).

A nucleic acid molecule that encodes such a second/fourth polypeptide chain is (SEQ ID NO: 32):

gaaattgtcc tgacacagtc tcccgcaacc ctgagtttga
gtcctgggga gcgagcaact ctctcctgcc gagcctccca
gagtatctcc tcctacctcg cctggtacca acagaagcca
gggcaggctc caaggctgct tatctatgac gcctctaacc
gcgcaactgg gattcccgca cgcttctccg gctctggttc
cggcacagac tttacactta ctatctctag cctggagcca
gaagactttg ccgtgtacta ttgtcagcaa cgttccaatt
ggccccttac ctttgggcag ggcactaact tggaaatcaa
aggtggcgga tccggcggcg gaggccaggt tcagctggtc
gagagtggtg gcggcgttgt gcaacctggg cgttccctcc
gattggactg taaagcttcc ggcattactt tctcaaattc
cggcatgcat tgggtgaggc aagcccctgg aaaagggctc
gaatgggtgg ctgtgatttg gtacgatggc agcaaacggt
actacgccga ttctgttaag ggccgcttta ccatctcccg
cgataactca aagaacacac tgtttctgca aatgaatagt
cttagagccg aggacaccgc cgtgtactac tgtgccacaa
atgacgatta ttgggggcag ggcacattgg tcacagtgtc
ttccggagga tgtggcggtg gaaaagtggc cgcatgtaag
gagaaagttg ctgctttgaa agagaaggtc gccgcactta
aggaaaaggt cgcagccctg aaagag

### III. Pharmaceutical Compositions

The present invention includes pharmaceutical compositions for the treatment of a cancer or a disease associated with the presence of a pathogen. Such compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a modified PD-1×LAG-3 bi-specific diabody of the present invention, (and especially a PD-1×LAG-3 bi-specific, tetra-valent, Fc-DART® diabody of the present invention) and a pharmaceutically acceptable carrier. Preferably, compositions of the invention comprise a prophylactically or therapeutically effective amount of one or more molecules of the invention and a pharmaceutically acceptable carrier. The invention also encompasses pharmaceutical compositions comprising such modified diabodies and a second therapeutic antibody that is specific for a particular pathogen-associated antigen, and a pharmaceutically acceptable carrier.

As used herein, the term “cancer” refers to a disease characterized by the presence of a malignant tumor. Such cancers include an adrenal gland cancer, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phacochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, or a uterine cancer.

As used herein, the term “disease associated with the presence of a pathogen” refers to a disease associated with an infection by a bacterium (e.g., E. coli, C. difficile, Salmonella thyphimurium, Pseudomonas aeruginosa, Vibrio cholerae, Neisseria gonorrhoeae, Helicobacter pylori, Hemophilus influenzae, Shigella dysenteriae, Staphylococcus aureus, Mycobacterium tuberculosis and Streptococcus pneumonia, etc.), a fungus (e.g., Candida, Aspergillus, Cryptococcus, Coccidioides, Histoplasma, Pneumocystis, Stachybotrys, etc.), a protozoan (Amocbozoa, Excavata, Chromalveolata, Entamoeba, Plasmodium, Giardia, Trypanosoma, Coccidia, Besnoitia, Dicrocoelium, Leishmania, etc.) or a virus (and especially an adenovirus, an adeno-associated virus, a B virus (macacine herpesvirus I), a BK virus, a bunyavirus, a chikungunya virus, a cocksackie virus, a coronavirus, a cytomegalovirus, an castern equine encephalitis virus, an ebola virus, an enterovirus, an Epstein-Barr virus, a hantavirus, a hepatitis A virus, a hepatitis B virus, a hepatitis C virus, a hepatitis D virus, a hepatitis E virus, a herpes simplex virus 1, a herpes simplex virus 2, a human foamy virus, a human herpes virus 3, a human herpes virus 5, a human herpes virus 6, a human herpes virus 7, a human immunodeficiency virus, a human papillomavirus, a human ß-lymphotropic virus, a human T-cell leukemia virus I, a human T-cell leukemia virus II, an influenza virus, a JC virus, a JEV, a Kaposi's sarcoma-associated herpesvirus, a Lassa virus, a lymphocytic choriomenengitis virus, a Marburg virus, a measles virus, a mumps virus, a Nipah virus, a norovirus, a Norwalk virus, an orthoreovirus, a parainfluenza virus, a parvovirus, a poliovirus, a rabies virus, a reovirus, a respiratory syncytial virus, rhinovirus, a Rift Valley fever virus, a rotavirus, rubella virus, a smallpox virus, a St Louis encephalitis virus, a variola major virus, a variola minor virus, a vericella-zoster virus, a West Nile virus, a western equine encephalitis virus, or a yellow fever virus).

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) infected cells or other diseased cells, decreasing symptoms resulting from the disease, increasing the quality of life of those suffering from the disease, decreasing the dose of other medications required to treat the disease, delaying the progression of the disease, and/or prolonging survival of companion animal recipients.

In a specific embodiment, the term “pharmaceutically acceptable” means approved by a regulatory agency of the Federal or a state government or listed in the U.S. Pharmacopcia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, tricthylamine, 2-ethylamino ethanol, histidine, procaine, etc.

The invention also provides a pharmaceutical pack or kit comprising one or more containers containing a PD-1×LAG-3 bi-specific diabody of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more molecules of the invention. In another embodiment, a kit further comprises one or more other prophylactic or therapeutic agents useful for the treatment of cancer or a disease characterized by the presence of a pathogen-associated antigen, in one or more containers. In another embodiment, a kit further comprises one or more antibodies or diabodies that bind one or more pathogen-associated antigens. In certain embodiments, the other prophylactic or therapeutic agent is a chemotherapeutic. In other embodiments, the prophylactic or therapeutic agent is a biological or hormonal therapeutic.

### IV. Methods of Producing the PD-1×LAG-3 Bi-Specific Diabodies of the Present Invention

The PD-1×LAG-3 bi-specific diabodies of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco) or transgenic milk. Suitable methods for expressing antibodies recombinantly in plants or milk have been disclosed (see, for example, Pecters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., chimeric, humanized, single-chain, etc. are known in the art. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

The antibodies or protein of interest may be subjected to sequencing by Edman degradation, which is well-known to those of skill in the art. The peptide information generated from mass spectrometry or Edman degradation can be used to design probes or primers that are used to clone the protein of interest.

An alternative method of cloning the protein of interest is by “panning” using purified proteins or portions thereof for cells expressing the antibody or protein of interest. The “panning” procedure may be conducted by obtaining a cDNA library from tissues or cells that express or over-express the desired cDNAs in a second cell type, and screening the transfected cells of the second cell type for a specific binding to the desired protein. Detailed descriptions of the methods used in cloning mammalian genes coding for cell-surface proteins by “panning” can be found in the art (see, for example, Aruffo, A. et al. (1987) “Molecular Cloning Of A CD28 cDNA By A High-Efficiency COS Cell Expression System,” Proc. Natl. Acad. Sci. (U.S.A.) 84:8573-8577 and Stephan, J. et al. (1999) “Selective Cloning Of Cell Surface Proteins Involved In Organ Development: Epithelial Glycoprotein Is Involved In Normal Epithelial Differentiation,” Endocrinol. 140:5841-5854).

cDNAs encoding antibodies, and other peptide agonists, antagonists and modulators can be obtained by reverse transcribing the mRNAs from a particular cell type according to standard methods in the art. Specifically, mRNA can be isolated using various lytic enzymes or chemical solutions according to the procedures set forth in Sambrook et al. supra or extracted by commercially available nucleic-acid-binding resins following the accompanying instructions provided by manufacturers (e.g., Qiagen, Invitrogen, Promega). The synthesized cDNAs are then introduced into an expression vector to produce the antibody or protein of interest in cells of a second type. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, and cosmids.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose of isolating the genes encoding the antibody, polypeptide or protein of interest. Non-limiting examples of suitable mammalian host cells include but are not limited to COS, HeLa, and CHO cells. Preferably, the host cells express the cDNAs at a level of about 5-fold higher, more preferably 10-fold higher, more preferably 20-fold higher, more preferably 50-fold higher, more preferably 100-fold higher than that of the corresponding endogenous antibody or protein of interest, if present, in the host cells. Screening the host cells for a specific binding to a desired protein is preferably effected by an immunoassay or FACS. A cell over-expressing the antibody or protein of interest can be identified in this way.

Various techniques are also available which may now be employed to produce mutant peptide agonists, antagonists, and modulators which encodes for additions, deletions, or changes in amino acid sequence of the resultant protein relative to the parent peptide agonist, antagonist or modulator molecule.

The invention includes modifications to the PD-1×LAG-3 bi-specific diabody of the invention that do not significantly affect their properties and variants that have enhanced or decreased activity. Modification of polypeptides is routine practice in the art. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tyrosine. These polypeptides also include glycosylated and non-glycosylated polypeptides, as well as polypeptides with other post translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention also encompasses fusion proteins comprising one or more fragments or regions from the polypeptides and antibodies of this invention. In one embodiment, a fusion polypeptide is provided that comprises at least 10 contiguous amino acids of variable light chain region and at least 10 amino acids of variable heavy chain region. In another embodiment, the fusion polypeptide contains a heterologous immunoglobulin constant region. In another embodiment, the fusion polypeptide contains a Light Chain Variable Region and a Heavy Chain Variable Region of an antibody produced from a publicly-deposited hybridoma. For purposes of this invention, an antibody fusion protein contains one or more polypeptide Domains that specifically bind to a desired viral epitope or a desired activating receptor of an immune effector cell or a protein present on the surface of an immune effector cell that expresses such an activating receptor and another amino acid sequence to which it is not attached in the native molecule, for example, a heterologous sequence or a homologous sequence from another region.

The invention includes polypeptides comprising an amino acid sequence of the antibodies of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, such a polypeptide could be produced by an automated polypeptide synthesizer employing the solid phase method.

### V. Uses of the Compositions of the Invention

The present invention encompasses compositions, including pharmaceutical compositions, comprising the PD-1×LAG-3 bi-specific diabodies of the present invention, polypeptides derived from such molecules, polynucleotides comprising sequences encoding such molecules or polypeptides, and other agents as described herein.

In that the PD-1×LAG-3 bi-specific diabodies of the present invention have the ability attenuate the inhibition of the immune system mediated by PD-1 and LAG-3, the PD-1×LAG-3 bi-specific diabodies of the present invention may be used to treat any disease or condition associated with an undesirably suppressed immune system, including cancer and diseases that are associated with the presence of a pathogen (e.g., a bacterial, fungal, viral or protozoan infection).

### VI. Methods of Administration

The compositions of the present invention may be provided for the treatment, prophylaxis, and amelioration of one or more symptoms associated with a disease, disorder or infection by administering to a subject an effective amount of a pharmaceutical composition of the invention. In a preferred aspect, such compositions are substantially purified (i.e., substantially free from substances that limit its effect or produce undesired side-effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering the PD-1×LAG-3 bi-specific diabodies of the present invention include, but are not limited to, parenteral administration (e.g., intradermal, intramuscular, intraperitoneal, intravenous and subcutaneous), epidural, and mucosal (e.g., intranasal and oral routes). In a specific embodiment, the molecules of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985, 320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the PD-1×LAG-3 bi-specific diabodies of the present invention may be packaged in a hermetically sealed container such as an ampoule or sachette indicating the quantity of such molecules. In one embodiment, the PD-1×LAG-3 bi-specific diabodies of the present invention are supplied as a dry sterilized lyophilized powder or water free concentrate in a hermetically sealed container and can be reconstituted, e.g., with water or saline to the appropriate concentration for administration to a subject. Preferably, the PD-1×LAG-3 bi-specific diabodies of the present invention are supplied as a dry sterile lyophilized powder in a hermetically sealed container at a unit dosage of at least 5 μg, more preferably at least 10 μg, at least 15 μg, at least 25 μg, at least 50 μg, at least 100 μg, or at least 200 μg.

The lyophilized PD-1×LAG-3 bi-specific diabodies of the present invention should be stored at between 2 and 8° C. in their original container and the molecules should be administered within 12 hours, preferably within 6 hours, within 5 hours, within 3 hours, or within 1 hour after being reconstituted. In an alternative embodiment, the PD-1×LAG-3 bi-specific diabodies of the present invention are supplied in liquid form in a hermetically sealed container indicating the quantity and concentration of the molecule, fusion protein, or conjugated molecule. Preferably, the liquid form of the PD-1×LAG-3 bi-specific diabodies of the present invention is supplied in a hermetically sealed container in which the molecules are present at a concentration of least 1 μg/ml, more preferably at least 2.5 μg/ml, at least 5μg/ml, at least 10 μg/ml, at least 50 μg/ml, or at least 100 μg/ml.

As used herein, an “effective amount” of a pharmaceutical composition, in one embodiment, is an amount sufficient to effect beneficial or desired results including, without limitation, clinical results such as decreasing symptoms resulting from the disease attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

An effective amount can be administered in one or more administrations. For purposes of this invention, an effective amount of drug, compound, or pharmaceutical composition is an amount sufficient to reduce the proliferation of (or the effect of) viral presence and to reduce and/or delay the development of the viral disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art. Typical dosages for antibody administration comprise one or more unit doses between 0.1-to 100 mg/kg/body weight.

The amount of the PD-1×LAG-3 bi-specific diabodies of the present invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For the PD-1×LAG-3 bi-specific diabodies of the present invention, the dosage administered to a patient is typically at least about 0.01 μg/kg, at least about 0.05 μg/kg, at least about 0.1 μg/kg, at least about 0.2 μg/kg, at least about 0.5 μg/kg, at least about 1 μg/kg, at least about 2 μg/kg, at least about 5 μg/kg, at least about 10 μg/kg, at least about 20 μg/kg, at least about 50 μg/kg, at least about 0.1 mg/kg, at least about 1 mg/kg, at least about 5 mg/kg, at least about 10 mg/kg, at least about 30 mg/kg, at least about 50 mg/kg, at least about 75 mg/kg, at least about 100 mg/kg, at least about 125 mg/kg, at least about 150 mg/kg or more of the subject's body weight.

In another embodiment, the patient is administered a treatment regimen comprising one or more doses of such prophylactically or therapeutically effective amount of the PD-1×LAG-3 bi-specific diabodies of the present invention, wherein the treatment regimen is administered over 2 days, 3 days, 4 days, 5 days, 6 days or 7 days. In certain embodiments, the treatment regimen comprises intermittently administering doses of the prophylactically or therapeutically effective amount of the PD-1×LAG-3 bi-specific diabodies of the present invention (for example, administering a dose on day 1, day 2, day 3 and day 4 of a given weck and not administering doses of the prophylactically or therapeutically effective amount of the PD-1×LAG-3 bi-specific diabodies of the present invention on day 5, day 6 and day 7 of the same weck). Typically, there are 1, 2, 3, 4, 5, or more courses of treatment. Each course may be the same regimen or a different regimen.

In another embodiment, the administered dose escalates over the first quarter, first half or first two-thirds or three-quarters of the regimen(s) (e.g., over the first, second, or third regimens of a 4 course treatment) until the daily prophylactically or therapeutically effective amount of the PD-1×LAG-3 bi-specific diabodies encompassed by the invention is achieved.

In one embodiment, the dosage of the PD-1×LAG-3 bi-specific diabodies of the present invention administered to a patient may be calculated for use as a single agent therapy. In another embodiment the PD-1×LAG-3 bi-specific diabodies of the present invention are used in combination with other therapeutic compositions and the dosage administered to a patient are lower than when such diabodies are used as a single agent therapy.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

In another embodiment, the compositions can be delivered in a vesicle, in particular a liposome (See Langer (1990) “New Methods Of Drug Delivery,” Science 249:1527-1533); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327;see generally ibid.).

In yet another embodiment, the compositions can be delivered in a controlled release or sustained release system. Any technique known to one of skill in the art can be used to produce sustained release formulations comprising one or more molecules of the invention. See, e.g., U.S. Pat. No. 4,526,938; PCT publication WO 91/05548; PCT publication WO 96/20698; Ning et al. (1996) “Intratumoral Radioimmunotheraphy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleck et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, (1987) “Implantable Pumps,” CRC Crit. Rev. Biomed. Eng. 14:201-240; Buchwald et al. (1980) “Long-Term, Continuous Intravenous Heparin Administration By An Implantable Infusion Pump In Ambulatory Patients With Recurrent Venous Thrombosis,” Surgery 88:507-516; and Saudek et al. (1989) “A Preliminary Trial Of The Programmable Implantable Medication System For Insulin Delivery,” N. Engl. J. Med. 321:574-579). In another embodiment, polymeric materials can be used to achieve controlled release of antibodies (see e.g., MEDICAL APPLICATIONS OF CONTROLLED RELEASE, Langer and Wise (cds.), CRC Pres., Boca Raton, Florida (1974); CONTROLLED DRUG BIOAVAILABILITY, DRUG PRODUCT DESIGN AND PERFORMANCE, Smolen and Ball (eds.), Wiley, New York (1984); Levy et al. (1985) “Inhibition Of Calcification Of Bioprosthetic Heart Valves By Local Controlled-Release Diphosphonate,” Science 228:190-192; During et al. (1989) “Controlled Release Of Dopamine From A Polymeric Brain Implant: In Vivo Characterization,” Ann. Neurol. 25:351-356; Howard et al. (1989) “Intracerebral Drug Delivery In Rats With Lesion-Induced Memory Deficits,” J. Neurosurg. 7 (1): 105-112); U.S. Pat. No. 5,679,377; U.S. Pat. No. 5,916,597; U.S. Pat. No. 5,912,015; U.S. Pat. Nos. 5,989,463; 5,128,326; PCT Publication No. WO 99/15154; and PCT Publication No. WO 99/20253). Examples of polymers used in sustained release formulations include, but are not limited to, poly(2-hydroxy ethyl methacrylate), poly (methyl methacrylate), poly (acrylic acid), poly (ethylene-co-vinyl acetate), poly (methacrylic acid), polyglycolides (PLG), polyanhydrides, poly (N-vinyl pyrrolidone), poly (vinyl alcohol), polyacrylamide, poly (ethylene glycol), polylactides (PLA), poly (lactide-co-glycolides) (PLGA), and polyorthoesters. In yet another embodiment, a controlled release system can be placed in proximity of the therapeutic target (e.g., the lungs), thus requiring only a fraction of the systemic dose (scc, e.g., Goodson, in MEDICAL APPLICATIONS OF CONTROLLED RELEASE, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ controlled release of the bioactive material from the polymer system. The implantation can generally occur anywhere within the body of the patient in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled release systems are discussed in the review by Langer (1990, “New Methods Of Drug Delivery,” Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al. (1996) “Intratumoral Radioimmunotheraphy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleck et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety.

In a specific embodiment where the composition of the invention is a nucleic acid encoding one or more of the polypeptide chains of a PD-1×LAG-3 bi-specific diabody of the present invention, the nucleic acid can be administered in vivo to promote expression of its encoded diabody, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (Sec e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of the PD-1×LAG-3 bi-specific diabodies of the present invention can include a single treatment or, preferably, can include a series of treatments. In a preferred example, a subject is treated with molecules of the invention one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the molecules used for treatment may increase or decrease over the course of a particular treatment.

Having now generally described the invention, the same will be more readily understood through reference to the following Examples. Such Examples are provided by way of illustration and are not intended to be limiting of the present invention unless specified.

### Example 1

### Production and Properties of PD-1×LAG-3 Bi-Specific Diabodies

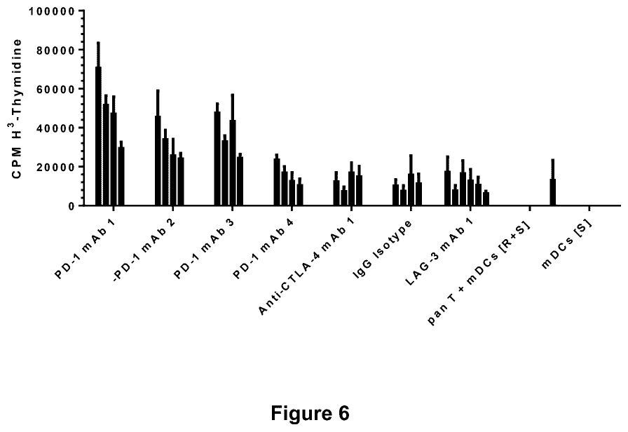

Within the context of the allo-MLR assay, T-cells are induced to proliferate in response to HLA-mismatching (Latchman, Y. E. et al. (2004) “PD-L1-Deficient Mice Show That PD-L1 On T-Cells, Antigen-Presenting Cells, And Host Tissues Negatively Regulates T-Cells.” Proc. Natl. Acad. Sci. (U.S.A.) 101(29):10691-10696; Wang, W. et al. (2008) “PD-L1/PD-1 Signal Deficiency Promotes Allogeneic Immune Responses And Accelerates Heart Allograft Rejection,” Transplantation 86(6):836-44) or mitogenic/pharmacological stimulation. Agonist antibodies that target costimulatory molecules are known to induce proliferative responses by re-enforcing T-cell signaling and stabilizing transcription factors that promote or drive T-cell effector function (Melero, I. et al. (2013) “Agonist Antibodies to TNFR Molecules That Costimulate T and NK Cells,” Clin. Cancer Res. 19(5):1044-1053). Similarly, antagonist antibodies that target key checkpoint molecules that negatively regulate T-cell responses can induce proliferative responses by maintaining T-cell signaling and effector function and thereby improving antitumor immunity (Capece, D. et al. (2012) “Targeting Costimulatory Molecules to Improve Antitumor Immunity,” J. Biomed. Biotech. 2012:926321). The effect of monoclonal antibodies against co-stimulatory or checkpoint targets on proliferation in response to alloantigen can be easily measure in short-term mixed lymphocyte (allo-MLR) reactions by following the incorporation of 3H-thymidine. To address ability of antibodies against checkpoint inhibitors to enhance proliferation, benchmark anti-PD-1 or anti-LAG-3 mAbs were gencrated, purified, and exogenously added at the initiation of allo-MLR assay at 20, 10, 5, 2.5, and 1.25 μg/ml (FIG. 5). At the end of 5-6 days, the 96-well plated was pulse with 3H-thymidine and cultured for 18 hrs to measure proliferation. Several benchmark antibodies against human PD-1, LAG-3, and CTLA-4 were evaluated in their capacity to enhance T-cell proliferation in response to allo-antigen stimulation. As shown in FIG. 6, the addition of PD-1 mAb 1 (5C4 (BMS-936558), PD-1 mAb 2 (MK-3475; Merck, lambrolizumab), or PD-1 mAb 3 (EH12.2H7; Dana Farber) at the start of the allo-MLR assay, induced strong T-cell proliferation compared to IgG1 isotype control antibody or the control wells containing responders and stimulators. Wells containing irradiated stimulator cells alone demonstrated no proliferation. Although a dose dependent proliferative response was observed, PD-1 mAb 4 (CT-011; CureTech, BAT-1) showed minimal proliferation compared to PD-1 mAb 1 (5C4 (BMS-936558), PD-1 mAb 2 (MK-3475; Merck, lambrolizumab), or PD-1 mAb 3 (EH12.2H7; Dana Farber). A slight dose dependent proliferative response was also observed with LAG-3 mAb 1 (25F7; BMS-986016, Medarex/BMS), which compared similarly to Yervoy® ipilimumab, an anti-CTLA-4 mAb (Bristol Myers-Squib).

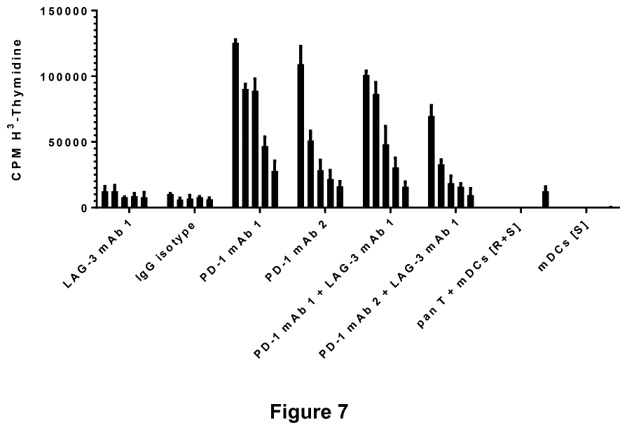

The striking effectiveness of PD-1 mAb 1 (5C4 (BMS-936558), given concurrently with LAG-3 mAb 1 (25F7; BMS-986016, Medarex/BMS) in eliciting potent anti-tumor immunity in animal models when compared to either antibody given alone (Woo, S. R. et al. (2012) “Immune Inhibitory Molecules LAG-3 And PD-1 Synergistically Regulate T-Cell Function To Promote Tumoral Immune Escape,” Cancer Res. 72 (4): 917-927), suggested that benchmark mAbs given in combination may potentiate allo-induced T-cell proliferation greater than either antibody alone. As shown in FIG. 7, anti-LAG-3 mAb (25F7) was evaluated for its proliferative potential either alone or in combination with the anti-PD-1 mAbs (PD-1 mAb 1 (5C4 (BMS-936558) or PD-1 mAb 2 (MK-3475; Merck, lambrolizumab)). As observed previously, both anti-PD-1 mAbs induced potent proliferation in a dose dependent manner. In contrast, provision of anti-LAG-3 mAb with either anti-PD-1 mAb did not induce enhanced proliferation beyond that observed with anti-PD-1 mAbs alone. Anti-LAG-3 mAb alone exhibited only slight T-cell proliferation in comparison to isotype IgG1 control or responder plus stimulator control wells.

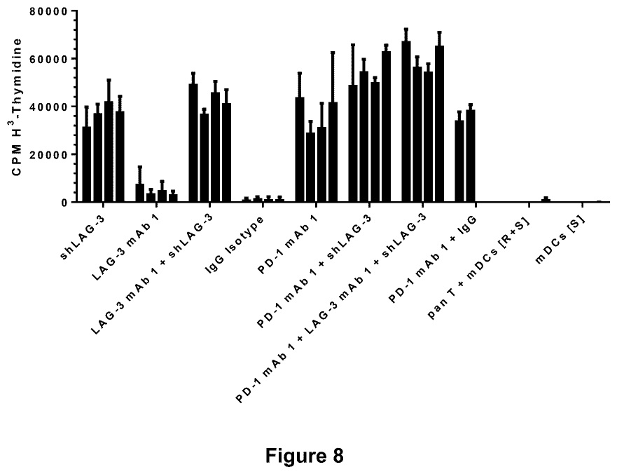

The inability of anti-LAG-3 mAbs to induce proliferation alone or in combination beyond that observed with anti-PD-1 mAbs suggested that either LAG-3 expression is absent on T-cells during the allo-MLR assay or that anti-LAG-3 mAb does not bind to LAG-3 to block a negative signal cascade. In order to assay the potential to induce LAG-3 signal transduction, soluble human LAG-3 protein (“shLAG-3”) was added to the allo-MLR and compared against anti-LAG-3 mAb and/or anti-PD-1 mAbs. As shown in FIG. 8, soluble human LAG-3, which binds to human HLA-class II molecules expressed on both APCs and CD4 T-cells, induced a robust proliferative response compared to IgG isotype or responder plus stimulator control wells. Addition of LAG-3 mAb 1 (25F7; BMS-986016, Medarex/BMS), did not seem to block the proliferative effect of soluble human LAG-3 protein and may have slightly enhanced T-cell proliferation, as a slight dose dependent proliferative response was observed. Consistent with previous observations, anti-PD-1 mAb induced potent T-cell proliferation that was further enhanced by the addition of both soluble human LAG-3 protein and/or LAG-3 mAb 1 (25F7; BMS-986016, Medarex/BMS). Addition of isotype IgG1 control to anti-PD-1 antibodies did not enhance T-cell proliferation beyond that observed with PD-1 mAb 1 (5C4 (BMS-936558) alone. The ability of soluble LAG-3 to induce potent T-cell proliferation even in the presence of anti-LAG-3 mAbs is unclear. One possibility is that anti-LAG-3 mAb is simply unable to block the strong proliferative signal induced by soluble LAG-3 mAb. An alternative—but not necessarily non-mutually exclusive possibility—is that soluble human LAG-3 protein together with anti-LAG-3 forms immune cross-linking complexes that can further potentiate proliferative responses.

The ability of soluble LAG-3 to potentiate T-cell proliferation suggested that introduction of both anti-PD-1 and anti-LAG-3 mAbs in close proximity might enhance T-cell proliferative responses within the allo-MLR assay. To address this possibility, benchmark anti-PD-1 and anti-LAG-3 mAbs were constructed within the dual affinity retargeting (DART®)-bi-specific format in two orientations: a LAG-3 mAb 1 (25F7; BMS-986016, Medarex/BMS)—PD-1 mAb 1 (5C4 (BMS-936558) bi-specific, tetra-valent Fc-DART® diabody (PD-1×LAG-3 Fc-DART®-1) and a PD-1 mAb 1 (5C4 (BMS-936558)—LAG-3 mAb 1 (25F7; BMS-986016, Medarex/BMS) bi-specific, tetra-valent Fc-DART® diabody (PD-1×LAG-3 Fc-DART®-1). Both Fc-DART® formats were exogenously added (in the dose-dependent manner described above) at the beginning of the allo-MLR and evaluated for their T-cell proliferative potential.

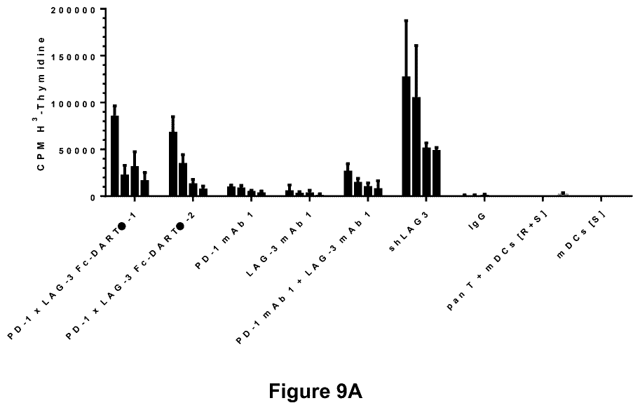

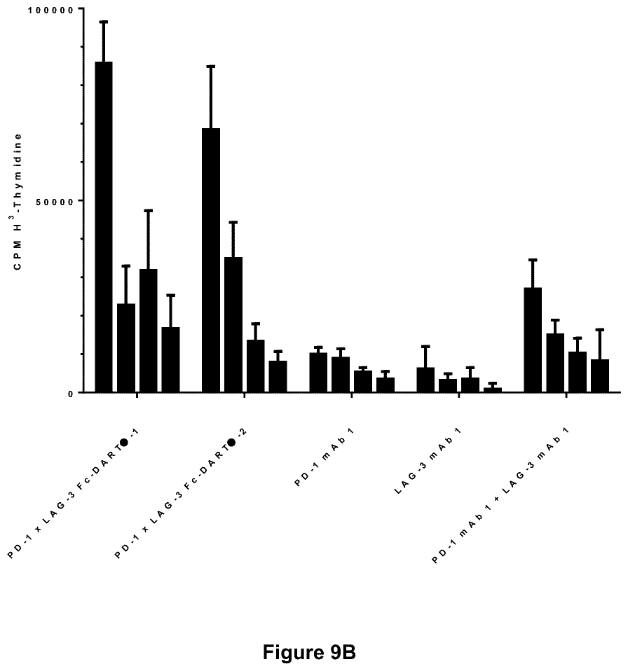

As shown in FIGS. 9A-9B, both-DART® diabodies induced surprisingly more potent T-cell proliferative responses than those obtained with PD-1 mAb 1 (5C4 (BMS-936558) and/or LAG-3 mAb 1 (25F7; BMS-986016, Medarex/BMS). Soluble human LAG-3 (“shLAG-3”) again demonstrated strong T-cell proliferation within the allo-MLR assay. The strong proliferative signal induced by both DART®s and soluble human LAG-3 protein, minimized the contribution of anti-PD-1 mAb or anti-LAG-3 mAb alone. While both anti-PD-1 and LAG-3 mAbs induced T-cell proliferation in a dose dependent manner beyond that observed with IgG1 isotype control or responder plus stimulator wells alone, the combination of anti-PD-1 with anti-LAG-3 demonstrated enhanced proliferation then either antibody alone, suggesting as previous reports in the literature have demonstrated a functional synergy (Wang, W. et al. (2008) “PD-L1/PD-1 Signal Deficiency Promotes Allogeneic Immune Responses And Accelerates Heart Allograft Rejection,” Transplantation 86(6):836-44; Melero, I. et al. (2013) “Agonist Antibodies to TNFR Molecules That Costimulate T and NK Cells,” Clin. Cancer Res. 19(5):1044-1053; Capece, D. et al. (2012) “Targeting Costimulatory Molecules to Improve Antitumor Immunity,” J. Biomed. Biotech. 2012:926321).

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A bi-specific Fc diabody capable of immunospecific binding to an epitope of PD-1 and to an epitope of LAG-3, wherein said diabody comprises four polypeptide chains, each having an amino terminus and a carboxy terminus, and wherein:
(A) said first and second polypeptide chains are covalently bonded to one another, said second and third polypeptide chains are covalently bonded to one another, and said third and fourth polypeptide chains are covalently bonded to one another;
(B) said first and third polypeptide chains of said diabody each comprise, in the N-terminal to C-terminal direction, a Light Chain Variable Domain of an antibody that is immunospecific for PD-1 or LAG-3, a Heavy Chain Variable Domain of an antibody that is immunospecific for LAG-3 or PD-1, a Heterodimer-Promoting Domain and a CH2-CH3 Domain, wherein said Light Chain Variable Domains and said Heavy Chain Variable Domains are incapable of associating to form an Epitope-Binding Site capable of binding an epitope of PD-1 or an epitope of LAG-3; and
(C) said second and said fourth polypeptide chains of said diabody each comprise, in the N-terminal to C-terminal direction, a Light Chain Variable Domain of an antibody that is immunospecific for PD-1 or LAG-3, a Heavy Chain Variable Domain of an antibody that is immunospecific for LAG-3 or PD-1, and a Heterodimer-Promoting Domain, wherein said Light Chain Variable Domains and said Heavy Chain Variable Domains are incapable of associating to form an Epitope-Binding Site capable of binding an epitope of PD-1 or an epitope of LAG-3;
and wherein:
(I) (1) said Light Chain Variable Domain of said first polypeptide chain and said Heavy Chain Variable Domain of said second polypeptide chain associate to form a first Epitope-Binding Site and said Heavy Chain Variable Domain of said first polypeptide chain and said Light Chain Variable Domain of said second polypeptide chain associate to form a second Epitope-Binding Site; and
(2) said Light Chain Variable Domain of said third polypeptide chain and said Heavy Chain Variable Domain of said fourth polypeptide chain associate to form a third Epitope-Binding Site and said Heavy Chain Variable Domain of said third polypeptide chain and said Light Chain Variable Domain of said fourth polypeptide chain associate to form a fourth Epitope-Binding Site;
wherein two of said formed Epitope-Binding Sites are capable of immunospecifically binding to an epitope of PD-1 and two of said formed Epitope-Binding Sites are capable of immunospecifically binding to an epitope of LAG-3;
II. said Heterodimer-Promoting Domains of said first and third polypeptide chains differ and have an amino acid sequence selected from the group consisting of: SEQ ID NO:16 and SEQ ID NO:17; and
III. said CH2-CH3 Domains of said first and third polypeptide chains associate to form an Fc Domain.

2. The bi-specific Fc diabody of claim 1, wherein said CH2-CH3 Domains of said first and third polypeptide chains each have the amino acid sequence of SEQ ID NO: 24.

3. The bi-specific Fc diabody of any of claim 1 or 2, wherein said Heavy Chain Variable Domain of an antibody that is immunospecific for LAG-3 has the amino acid sequence of SEQ ID NO:11, and wherein said Light Chain Variable Domain of an antibody that is immunospecific for LAG-3 has the amino acid sequence of SEQ ID NO:12.

4. The bi-specific Fc diabody of any of claims 1-3, wherein said Heavy Chain Variable Domain of an antibody that is immunospecific for PD-1 has the amino acid sequence of SEQ ID NO:2, and wherein said Light Chain Variable Domain of an antibody that is immunospecific for PD-1 has the amino acid sequence of SEQ ID NO:3.

5. A pharmaceutical composition that comprises an effective amount of the bi-specific Fc diabody of any of claims 1-4, and a pharmaceutically acceptable carrier.

6. The pharmaceutical composition of claim 5, wherein said effective amount of said bi-specific Fc diabody is an amount effective to treat cancer in a recipient individual in need of such treatment.

7. The pharmaceutical composition of claim 5, wherein said cancer is an adrenal gland cancer, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, or a uterine cancer.

8. The pharmaceutical composition of claim 5, wherein said effective amount of said bi-specific Fc diabody is an amount effective to treat a disease associated with the presence of a pathogen in a recipient individual in need of such treatment.

9. The pharmaceutical composition of claim 8, wherein said pathogen is a bacterium a fungus or a virus.

10. A method of treating cancer which comprises administering an effective amount of the pharmaceutical composition of any of claims 6-7 to an individual in need thereof.

11. A method of treating a disease associated with the presence of a pathogen which comprises administering an effective amount of the pharmaceutical composition of any of claims 8-9 to an individual in need thereof.
